(19) United States
(12) Patent Application Publication
(10) Pub. No.: US 2014/0361189 A1
(43) Pub. Date: Dec. 11, 2014

(54) RADIATION IMAGING SYSTEM

(71) Applicant: CANON KABUSHIKI KAISHA, Tokyo (JP)

(72) Inventors: Toshio Kameshima, Kumagaya-shi (JP); Tomoyuki Yagi, Honjo-shi (JP); Katsuro Takenaka, Honjo-shi (JP); Sho Sato, Saitama-shi (JP); Atsushi Iwashita, Saitama-shi (JP); Eriko Sato, Tokyo (JP); Hideyuki Okada, Honjo-shi (JP); Takuya Ryu, Kokubunji-shi (JP)

(73) Assignee: CANON KABUSHIKI KAISHA, Tokyo (JP)

(21) Appl. No.: 14/290,479

(22) Filed: May 29, 2014

(30) Foreign Application Priority Data
Jun. 5, 2013 (JP) 2013-118994
Jul. 9, 2013 (JP) 2013-143815

(51) Int. Cl. G01T 1/29

(52) U.S. Cl. CPC G01T 1/2928
USPC 250/393; 250/394

(57) ABSTRACT

A radiation imaging system includes: a sensor unit including a plurality of pixels arranged in a matrix, and configured to convert a radiation into an electric charge and to output a pixel output value; a detecting unit configured to detect an irradiation start of the radiation; and a control unit configured to output image information based on the irradiation of the radiation and to output dark image information for the plurality of pixels.

### BACKGROUND OF THE INVENTION

1. Field of the Invention

The present invention relates to a radiation imaging system suitably used for still picture imaging, such as general imaging, and for moving picture imaging, such as fluoroscopic imaging, in medical diagnosis.

2. Description of the Related Art

Currently, a radiation imaging apparatus using a plane sensor (Flat Panel Detector, hereinafter abbreviated as “FPD”) formed by a semiconductor material is widely used as an imaging apparatus used for medical image diagnosis and nondestructive inspection based on X-rays. The radiation imaging apparatus is used as, for example, a digital imaging apparatus for still picture imaging, such as general imaging, and for moving picture imaging, such as fluoroscopic imaging, in medical image diagnosis. An X-ray generating apparatus and the FPD are generally synchronized in the imaging apparatus. However, connection with the X-ray generating apparatus is necessary at the installation of the FPD, and there is a problem that the installation location is limited.

In this regard, Japanese Patent Application Laid-Open No. 2011-249891 discloses a technique of sensing an irradiation start of radiation based on a current flowing in a bias line of a conversion element, without constructing an interface with a radiation generating apparatus. Specifically, non-adjacent rows are sequentially scanned to perform reset operation, and when irradiation of a radiation is detected, the reset operation is stopped to shift the operation to accumulating operation. When the irradiation of the radiation is finished, scanning is sequentially performed, and read out operation of image data is performed. Japanese Patent Application Laid-Open No. 2011-249891 further discloses a technique of reading out offset correction data after the read out operation of the image data, at the same timing as the timing from the reset operation to the read out operation, wherein the radiation is not irradiated.

In Japanese Patent Application Laid-Open No. 2008-259045, an accumulation time of offset correction data is calculated based on a time from application of bias to radiation imaging, an accumulation time and a response characteristic of a dark current, and the offset correction data is read out based on the calculated accumulation time to correct a dark current response of a conversion element. A scanning time of each row at the acquisition of an offset signal after the X-ray imaging is calculated by arithmetic operation, and each row is scanned based on the calculated scanning time. Furthermore, dark current components to be superimposed on image data in radiation imaging are calculated from the time from the application of bias to the radiation imaging, the accumulation time, the response characteristic of the dark current and the offset correction data acquired in advance, in order to correct the dark current response of the conversion element.

The present inventors have found out that when the dark current components of the conversion element temporally vary, an artifact with level difference generated before and after a row in which the radiation is detected may not be corrected, and the image quality may be degraded in the technique of Japanese Patent Application Laid-Open No. 2011-249891. Particularly, when the X-ray is detected while repeating reset operation (dummy reading) called interlace scanning for separately scanning non-adjacent rows, such as even rows and odd rows, a stripe artifact of even and odd rows generated in the row direction may not be corrected, and the image quality may be degraded.

Meanwhile, Japanese Patent Application Laid-Open No. 2008-259045 does not have a concept of performing the accumulating operation after stopping the scanning when a radiation is detected in the middle of the reset operation (dummy reading) and a concept of sequentially scanning non-adjacent rows to perform the reset operation. Therefore, sufficient correction cannot be performed. As a result, an artifact generated before and after the detected row and a stripe artifact of even and odd rows generated in the row direction may degrade the image quality in Japanese Patent Application Laid-Open NO. 2008-259045, as in Japanese Patent Application Laid-Open No. 2011-249891. Furthermore, control of a drive unit based on the scanning time of each row calculated by arithmetic operation may be difficult, because a complicated circuit configuration is necessary.

### SUMMARY OF THE INVENTION

An object of the present invention is to provide a radiation imaging system that can prevent noise due to reset operation and that can obtain image information with excellent image quality.

According to an aspect of the present invention, a radiation imaging system comprising: a sensor unit including a plurality of pixels arranged in a matrix, and configured to convert a radiation into an electric charge and to output a pixel output value; a detecting unit configured to detect an irradiation start of the radiation; a control unit to perform (A) a first reset operation (A1) for resetting the plurality of pixels according to an interlace scanning until the detection of the irradiation start of the radiation by the detecting unit, a first accumulating operation (A2) for accumulating the electric charge in response to the detection of the irradiation start of the radiation by the detecting unit by stopping the first reset operation, and, thereafter, a first read out operation (A3) for reading out a pixel output value of the plurality of pixels in response to an irradiation end of the radiation so as to output an image information based on the irradiation of the radiation, and to further perform (B) a second reset operation (B1) for resetting the plurality of pixels according to the interlace scanning after the first read out operation, a second accumulating operation (B2) for accumulating the electric charge by stopping the second reset operation and a second read out operation (B3) for reading out the pixel output value so as to output a dark image information in accordance with the second accumulating operation, wherein the operations (A1), (A2), (A3), (B1), (B2) and (B3) are performed in this order; a first correcting unit configured to perform a correction by subtracting the dark image information from the image information based on the irradiation of the radiation; a second correcting unit configured to perform a correction of the image information after the correction by the first correcting unit, so as to correct (1) a level shift formed in the image information between an odd row and an even row of the pixels due to the interlace reset operation and to correct (2) a level shift formed in the image information between a row of the pixels scanned before stopping the interlace reset operation and a row of the pixels scanned after stopping the interlace reset operation.

Further features of the present invention will become apparent from the following description of exemplary embodiments with reference to the attached drawings.

### BRIEF DESCRIPTION OF THE DRAWINGS

FIG. 1 is a block diagram illustrating a configuration example of a radiation imaging system.

FIG. 2 is a circuit diagram illustrating a configuration example of a flat panel detector.

FIG. 3 is a flow chart illustrating operation of the radiation imaging system.

FIG. 4 is a drive timing diagram of the radiation imaging system.

FIG. 5 is a drive timing diagram of the radiation imaging system.

FIG. 6 is a diagram for describing electric charge accumulation periods.

FIGS. 7A and 7B are diagrams for describing a first offset correction.

FIGS. 8A and 8B are diagrams for describing a second offset correction.

FIGS. 9A and 9B are diagrams for describing the second offset correction.

FIGS. 10A and 10B are explanatory diagrams of dummy pixels.

FIG. 11 is a diagram for describing an artifact correction.

FIG. 12 is a diagram illustrating a configuration example of a radiation imaging system according to a second embodiment.

FIG. 13 is a flow chart illustrating operation of the radiation imaging system.

FIGS. 14A, 14B and 14C are diagrams for describing electric charge accumulation periods.

FIG. 15 is a diagram illustrating a configuration example of a radiation imaging system according to a third embodiment.

FIG. 16 is a diagram illustrating a configuration example of a radiation imaging system according to a fourth embodiment.

FIG. 17 is a flow chart illustrating operation of the radiation imaging system.

FIG. 18 is a drive timing diagram of the radiation imaging system.

FIG. 19 is a diagram for describing a correction method.

FIG. 20 is a diagram illustrating a configuration example of a radiation imaging system according to a fifth embodiment.

FIGS. 21A, 21B and 21C are diagrams for describing a prediction method.

FIG. 22 is a diagram illustrating a configuration example of a radiation imaging system according to a sixth embodiment.

FIGS. 23A and 23B are diagrams for describing a prediction method.

### DESCRIPTION OF THE EMBODIMENTS

Preferred embodiments of the present invention will now be described in detail in accordance with the accompanying drawings.

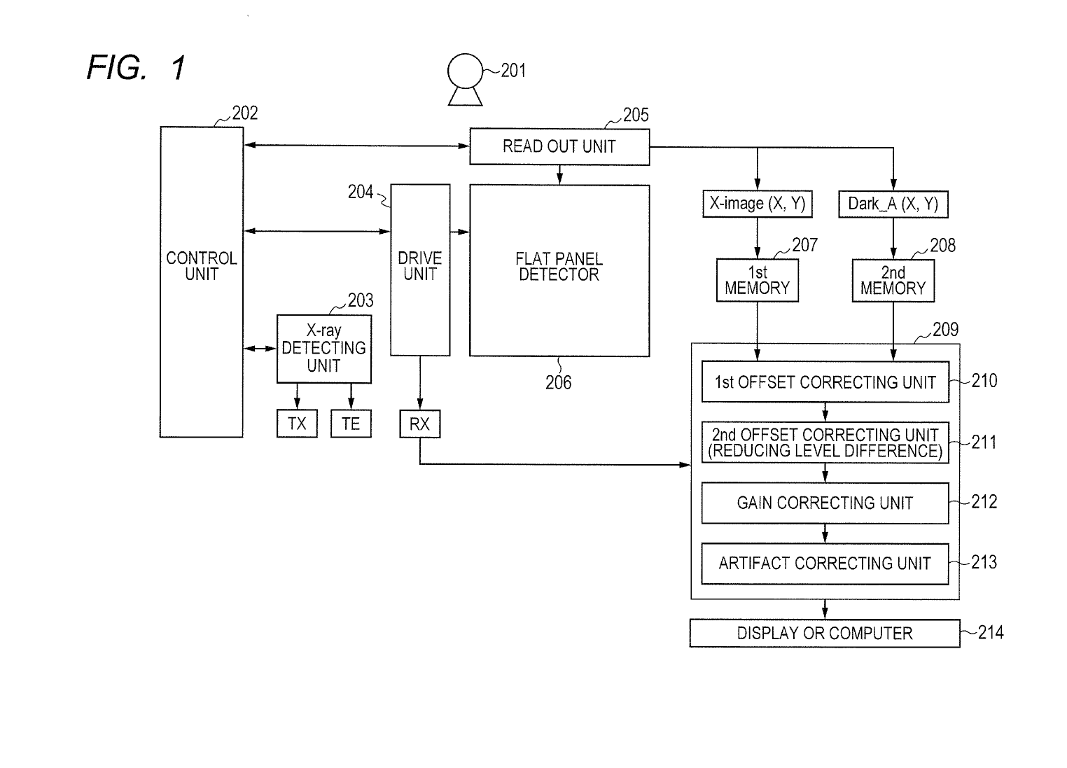

FIG. 1 is a block diagram illustrating a configuration example of a radiation imaging system according to an embodiment of the present invention. Radiations include α rays, β rays and γ rays that are beams created by particles (including photons) emitted by radioactive decay and include beams with equivalent or greater energy, such as X-rays, particle beams and cosmic rays. Hereinafter, an example in which the radiation is an X-ray will be described.

### First Embodiment

The radiation imaging system according to a first embodiment includes an X-ray generating apparatus 201, a control unit 202, an X-ray detecting unit 203, a drive unit 204, a read out unit 205, a flat panel detector 206, a first memory 207, a second memory 208, a correcting unit 209 and a display unit (or computer) 214. The correcting unit 209 includes a first offset correcting unit (first correcting unit) 210, a second offset correcting unit (second correcting unit) 211, a gain correcting unit 212 and an artifact correcting unit 213. The control unit 202 controls the X-ray detecting unit 203, the drive unit 204 and the read out unit 205. The drive unit 204 drives the flat panel detector 206.

The X-ray generating apparatus (radiation generating apparatus) 201 irradiates the flat panel detector 206 and the X-ray detecting unit 203 with an X-ray (radiation) through an object. The flat panel detector 206 includes a plurality of pixels arranged in a matrix of X columns×Y rows, and each pixel coverts the X-ray transmitted through the object to an electric charge, accumulates the electric charge, and outputs the accumulated electric charge as a pixel output value. The X-ray detecting unit 203 detects an irradiation start and an irradiation end of the X-ray and outputs an irradiation signal of the X-ray to the control unit 202. The flat panel detector 206 starts accumulating the electric charge based on the X-ray when the X-ray detecting unit 203 detects the irradiation start of the X-ray, and the flat panel detector 206 outputs the pixel output value based on the accumulated electric charge to the read out unit 205 when the X-ray detecting unit 203 detects the irradiation end of the X-ray. Consequently, the read out unit 205 outputs image information X-image (X, Y) based on the X-ray irradiation of the pixels of X columns×Y rows. The first memory 207 stores the image information X-image (X, Y).

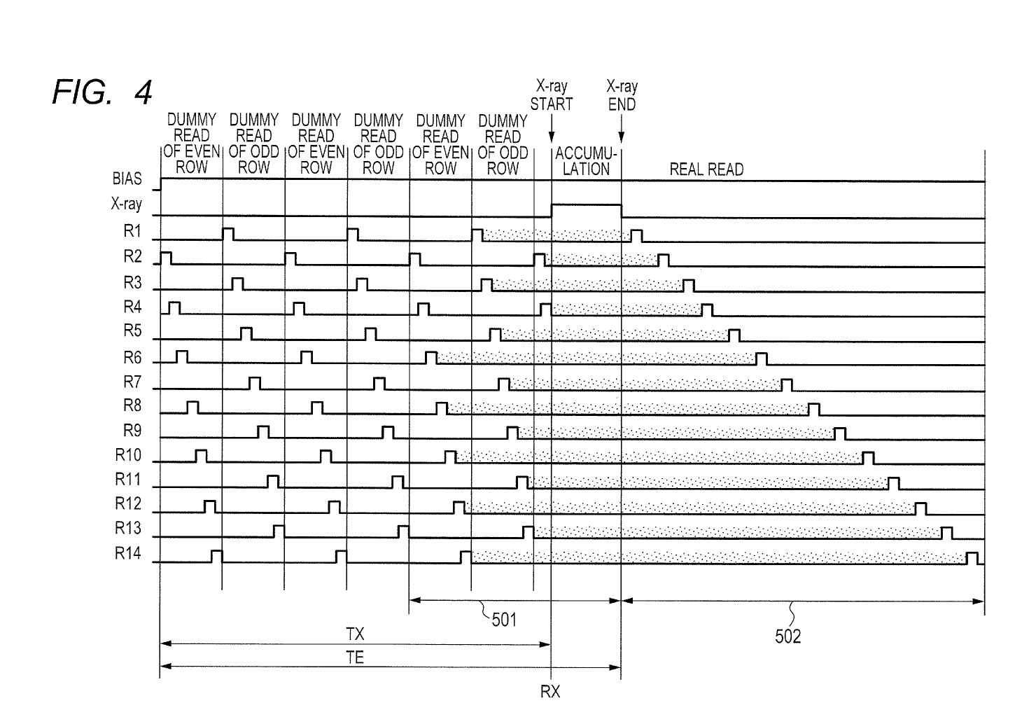

The X-ray detecting unit 203 outputs times TX and TE. The time TX is a time from a bias voltage application start time to an X-ray irradiation start time as illustrated in FIG. 4. The time TE is a time from the bias voltage application start time to an X-ray irradiation end time as illustrated in FIG. 4. The drive unit 204 outputs a row RX, at which dummy reading is stopped, to the correcting unit 209 at the X-ray irradiation start as illustrated in FIG. 4.

Thereafter, with the X-ray generating apparatus 201 not emitting the X-ray, the flat panel detector 206 performs the same operation (impersonation drive) again based on the periods TX, TE and the dummy reading stop row RX. The read out unit 205 outputs dark image information Dark_A (X, Y) of the pixels of X columns×Y rows. The second memory 208 stores the dark image information Dark_A (X, Y). The dark image information Dark_A (X, Y) is information related to offset components including fixed pattern noise of each pixel.

The first offset correcting unit 210 subtracts the dark image information Dark_A (X, Y) stored in the second memory 208 from the image information X-image (X, Y) stored in the first memory 207 to perform a first offset correction. The second offset correcting unit 211 applies a second offset correction to the image information corrected by the first offset correcting unit 210, through correction of an offset difference between an odd row and an even row, for example. The gain correcting unit 212 applies a gain correction to the image information corrected by the second offset correcting unit 211. The artifact correcting unit 213 corrects an artifact due to detection delay of the X-ray detecting unit 203, in the image information corrected by the gain correcting unit 212. The display unit (or computer) 214 displays (or processes) the image information corrected by the artifact correcting unit 213.

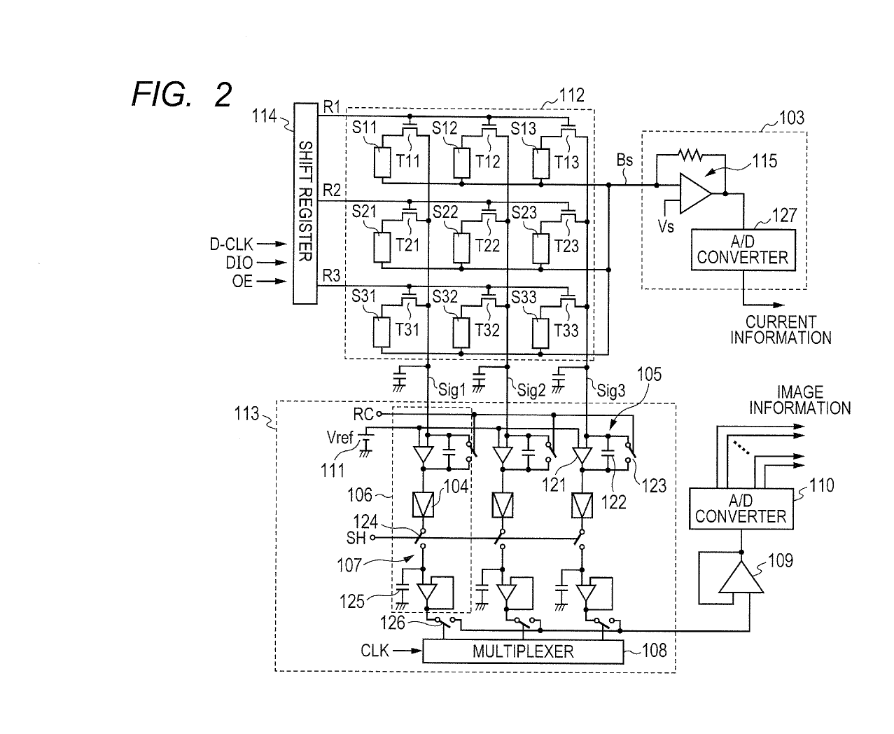

FIG. 2 is a circuit diagram illustrating a configuration example of the read out unit 205 and the flat panel detector 206 of FIG. 1. The flat panel detector 206 includes a vertical drive circuit 114, a sensor unit 112 and a bias power supply 103. The read out unit 205 includes a read out circuit 113, an output buffer amplifier 109 and an analog/digital (A/D) converter 110.

The flat panel detector 206 is a sensor including elements (pixels) for sensing radiation arranged in a two-dimensional matrix, and the flat panel detector 206 senses radiation and outputs image information. For the simplification of the description, FIG. 2 illustrates an example of the sensor unit 112 including pixels of 3 rows×3 columns. However, the actual flat panel detector 206 has more pixels and includes, for example, pixels of about 2800 rows×about 2800 columns in the case of 17 inches.

The sensor unit 112 includes a plurality of pixels arranged in a matrix. The pixels include conversion elements S11 to S33 for converting radiation or light to electric charges and switching elements T11 to T33 for outputting electrical signals based on the electric charges of the conversion elements S11 to S33, and the pixels output pixel output values. The conversion elements S11 to S33 are indirect-type conversion elements or direct-type conversion elements, and the conversion elements S11 to S33 convert the emitted radiation to electric charges. The indirect-type conversion elements S11 to S33 include wavelength converters for converting radiation to light and photoelectric conversion elements for converting the light to electric charges. The direct-type conversion elements S11 to S33 directly convert the radiation to electric charges. MIS photodiodes arranged on an insulating substrate, such as a glass substrate, and mainly made of amorphous silicon can be used as the photoelectric conversion elements for converting the emitted light to electric charges. The photoelectric conversion elements may be PIN photodiodes.

The switching elements T11 and T33 are transistors, each including a control terminal and two main terminals, and the switching elements T11 to T33 can be thin-film transistors (TFT). One of the electrodes of the conversion elements S11 to S33 is electrically connected to one of the two main terminals of the switching elements T11 to T33, and the other electrode is electrically connected to the bias power supply 103 through a common bias line Bs. The control terminals of the plurality of switching elements T11 to T13 of a first row are commonly and electrically connected to a drive line R1 of the first row. The control terminals of the plurality of switching elements T21 to T23 of a second row are commonly and electrically connected to a drive line R2 of the second row. The control terminals of the plurality of switching elements T31 to T33 of a third row are commonly and electrically connected to a drive line R3 of the third row. The vertical drive circuit 114 is, for example, a shift register, and the vertical drive circuit 114 supplies drive signals to the switching elements T11 to T33 through the drive lines R1 to R3 to control conductive states of the switching elements T11 to T33, row by row.

One of the main terminals of the plurality of switching elements T11 to T31 of the first column is connected to the conversion elements S11 to S31, and the other main terminal is electrically connected to a signal line Sig1 of a first column. While the switching elements T11 to T31 of the first column are conducted, electrical signals based on the electric charges of the conversion elements S11 to S31 of the first column are output to the read out circuit 113 through the signal line Sig1. One of the main terminals of the plurality of switching elements T12 to T32 of a second column are connected to the conversion elements S12 to S32, and the other main terminal is electrically connected to a signal line Sig2 of the second column. While the switching elements T12 to T32 of the second column are conducted, electrical signals based on the electric charges of the conversion elements S12 to S32 of the second column are output to the read out circuit 113 through the signal line Sig2. One of the main terminals of the plurality of switching elements T13 to T33 of a third column are connected to the conversion elements S13 to S33, and the other main terminal is electrically connected to a signal line Sig3 of the third column. While the switching elements T13 to T33 of the third column are conducted, electrical signals based on the electric charges of the conversion elements S13 to S33 of the third column are output to the read out circuit 113 through the signal line Sig 3. The plurality of signal lines Sig1 to Sig3 arranged in the column direction concurrently output, to the read out circuit 113, the electrical signals output from the plurality of pixels.

The read out circuit 113 includes amplifier circuits 106 that amplify the electrical signals of the signal lines Sig1 to Sig3, and the amplifier circuits 106 are arranged for each of the signal lines Sig1 to Sig3. Each of the amplifier circuits 106 includes an integrating amplifier 105, a variable gain amplifier 104 and a sample hold circuit 107. The integrating amplifiers 105 amplify the electrical signals of the signal lines Sig1 to Sig3. The variable gain amplifier 104 amplifies the electrical signal from the integrating amplifier 105 by a variable gain. The sample hold circuit 107 samples and holds the electrical signal amplified by the variable gain amplifier 104. The integrating amplifiers 105 include: arithmetic operation amplifiers 121 that amplify and output the electrical signals of the signal lines Sig1 to Sig3; integral capacities 122; and reset switches 123. The integrating amplifier 105 can change the value of the integral capacity 122 to change the gain (amplification factor). Inverting input terminals of the arithmetic operation amplifiers 121 of the columns are connected to the signal lines Sig1 to Sig3. Non-inverting input terminals are connected to a reference power supply 111 of a reference voltage Vref, and output terminals output the amplified electrical signals. The reference power supply 111 supplies the reference voltage Vref to the non-inverting input terminal of each of the arithmetic operation amplifiers 121. The integral capacity 122 is arranged between the inverting input terminal and the output terminal of the arithmetic operation amplifier 121. The sample hold circuit 107 includes: a sampling switch 124 of a control signal SH; and a sampling capacity 125. The read out circuit 113 further includes: switches 126 of the columns; and a multiplexer 108. The multiplexer 108 sequentially causes the switches 126 of the columns to enter the conductive state to sequentially output the electrical signals output in parallel from the amplifier circuits 106 to the output buffer amplifiers 109 as serial signals. The output buffer amplifier 109 converts the impedance of the electrical signals and outputs the electrical signals. The analog/digital (A/D) converters 110 convert analog electrical signals output from the output buffer amplifier 109 to digital electrical signals and outputs image information to one of the first memory 207 and the second memory 208 of FIG. 1.

The bias power supply 103 includes a current-voltage conversion circuit 115 and an A/D converter 127. The current-voltage conversion circuit 115 converts a current flowing in the bias line Bs to a voltage while supplying a bias voltage Vs to the bias line Bs and outputs the voltage to the A/D converter 127. The A/D converter 127 converts an analog voltage value including current information to a digital voltage value including the current information and outputs the digital voltage value. The X-ray detecting unit 203 of FIG. 1 uses the current information output by the A/D converter 127 to detect an irradiation start and an irradiation end of the X-ray.

The vertical drive circuit 114 outputs, to the drive lines R1 to R3, drive signals with conductive voltages for causing the switching elements T11 to T13 to enter the conductive state and drive signals with non-conductive voltage for causing the switching elements T11 to T33 to enter the non-conductive state according to control signals D-CLK, OE and DIO input from the drive unit 204 of FIG. 1. In this way, the vertical drive circuit 114 controls the conductive state and the non-conductive state of the switching elements T11 to T33 and drives the sensor unit 112. The control signal D-CLK is a shift clock of the shift register used as the vertical drive circuit 114. The control signal DIO is a transfer pulse of the shift register of the vertical drive circuit 114. The control signal OE is an output enable signal of the shift register of the vertical drive circuit 114. In this way, the vertical drive circuit 114 sets the drive time and the scanning direction. The drive unit 204 outputs a control signal RC, a control signal SH and a control signal CLK to the read out circuit 113 to control the operation of the constituent elements of the read out circuit 113. The control signal RC is a signal for controlling the operation of the reset switch 123 of the integrating amplifier 105. The control signal SH is a signal for controlling the sampling switch 124 of the sample hold circuit 107. The control signal CLK is a clock signal for controlling the operation of the multiplexer 108.

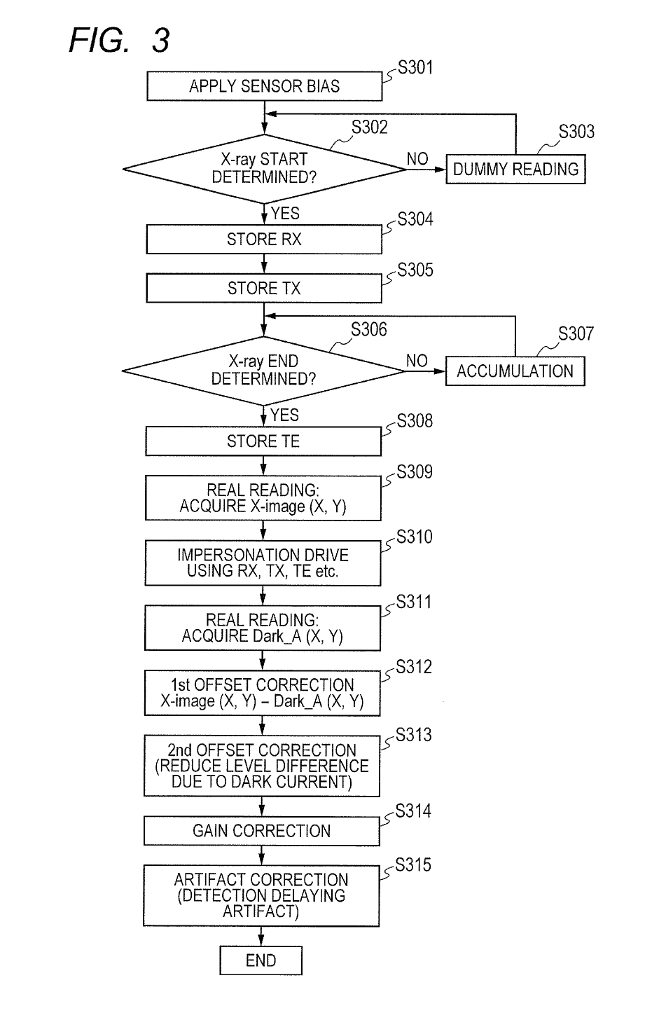

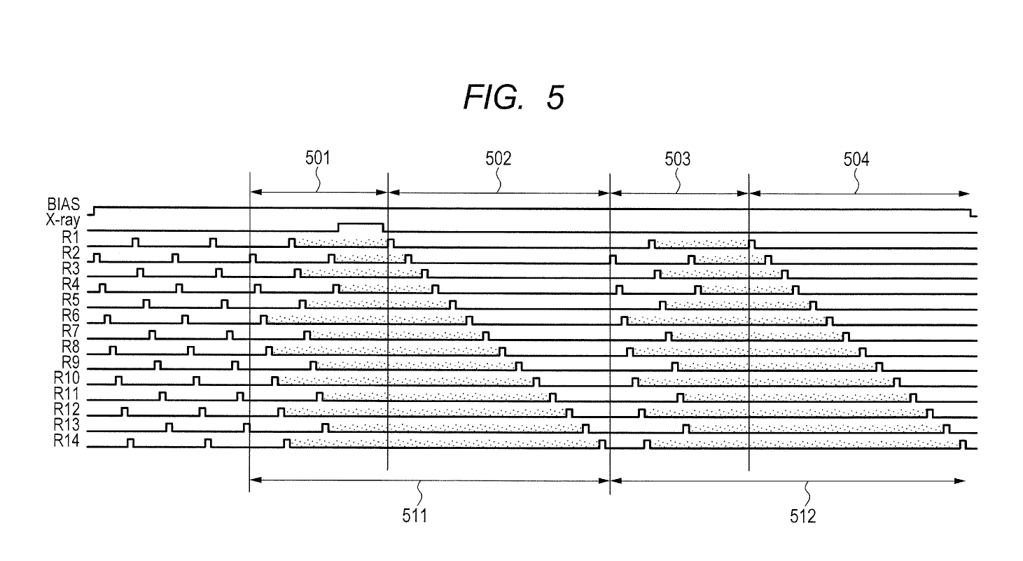

FIG. 3 is a flow chart illustrating a control method of the radiation imaging system of FIG. 1. FIG. 5 is a timing chart of the control method. FIG. 4 is an enlarged timing chart of part of FIG. 5. In step S301, the current-voltage conversion circuit 115 starts applying the bias voltage Vs to the bias line Bs as illustrated in FIG. 4.

In step S302, the control unit 202 determines whether irradiation with an X-ray is started. The X-ray detecting unit 203 outputs an X-ray irradiation signal to the control unit 202 if the current information (electrical signal based on the emitted X-ray) output by the A/D converter 127 is equal to or greater than a threshold. The control unit 202 determines that the irradiation with the X-ray is started if the X-ray irradiation signal is input and determines that the irradiation with the X-ray is not started if the X-ray irradiation signal is not input. The process proceeds to step S304 if the irradiation with the X-ray is started and proceeds to step S303 if the irradiation with the X-ray is not started. In step S303, the control unit 202 controls the sensor unit 112 to set the drive lines R1 to R14 to the conductive voltages and to cause the switching elements T11 to T33, etc., to enter the conductive state as illustrated in FIG. 4. In this way, reset operation (hereinafter, called “dummy reading”) of resetting the electric charges of the conversion elements S11 to S33, etc., generated by electric charge accumulation of a dark current is performed. Thereafter, the process returns to step S302. Before the irradiation with the X-ray, the sensor unit 112 repeats the reset operation of resetting the electric charges of the conversion elements S11 to S33, etc., caused by the dark current.

In the dummy reading of step S303, reset operation according to interlace scanning is performed as illustrated in FIG. 4. First, the drive lines R2, R4, R6 . . . and R14 of the pixels of the even rows are sequentially set to the conductive voltages. The switching elements T21, T41, etc., of the pixels of the even rows sequentially enter the conductive state. The electric charges of the conversion elements S21, S41, etc., of the pixels of the even rows are reset. Next, the drive lines R1, R3, R5 . . . and R13 of the pixels of the odd rows are sequentially set to the conductive voltages. The switching elements T11, T31, etc. of the pixels of the odd rows sequentially enter the conductive state. The electric charges of the conversion elements S11, S31, etc., of the pixels of the odd rows are reset. The operation of the set of the reset of the even rows and the reset of the odd rows is repeated until the detection of the start of the X-ray.

After the detection of the X-ray irradiation start, the control unit 202 stores the X-ray detected row RX through the drive unit 204 in step S304. The X-ray detected row RX denotes the row RX, at which the dummy reading is stopped due to the X-ray irradiation start, and is a fourth row corresponding to the drive line R4 in the case of FIG. 4.

In step S305, the control unit 202 stores the time TX through the X-ray detecting unit 203. As illustrated in FIG. 4, the time TX is a time from the bias voltage application start time to the X-ray irradiation start time.

In step S306, the X-ray detecting unit 203 determines whether the irradiation with the X-ray is finished. If the current information (electrical signal based on the emitted X-ray) output by the A/D converter 127 is smaller than the threshold, the X-ray detecting unit 203 determines the X-ray irradiation end and stops outputting the X-ray irradiation signal. The X-ray detecting unit 203 may stop outputting the X-ray irradiation signal after a lapse of a predetermined time (X-ray irradiation period) from the detection time of the X-ray irradiation start. The control unit 202 determines that the irradiation with the X-ray is finished if the input of the X-ray irradiation signal is stopped and determines that the irradiation with the X-ray is not finished if the X-ray irradiation signal is input. The process proceeds to step S308 if the irradiation with the X-ray is finished and proceeds to step S307 if the irradiation with the X-ray is not finished. In step S307, the control unit 202 controls the sensor unit 112 to perform accumulating operation of electric charges. Thereafter, the process returns to step S306. As illustrated in FIG. 4, the accumulating operation of the electric charges is operation of setting all of the drive lines R1 to R14 to the non-conductive voltages and causing the switching elements T11 to T33, etc., of all of the pixels to enter the non-conductive state to accumulate the electric charges based on the irradiation with the X-ray in the conversion elements S11 to S33, etc. The accumulating operation of the electric charges is performed in the conversion elements S11 to S33, etc., until the end of the irradiation with the X-ray.

After the detection of the X-ray irradiation end, the control unit 202 stores the time TE through the X-ray detecting unit 203 in step S308. As illustrated in FIG. 4, the time TE is a time from the bias voltage application start time to the X-ray irradiation end time.

In step S309, the control unit 202 controls the sensor unit 112 to perform real reading operation 502 of reading out the electric charges based on the irradiation with the X-ray. In the real reading operation 502, the drive lines R1 to R14 are sequentially caused to have pulses of the conductive voltages. The switching elements S11 to S33, etc., sequentially enter the conductive state, row by row. Electrical signals are sequentially output to the signal lines Sig1 to Sig3, etc., row by row, from the pixels of the top row to the pixels of the end row. The A/D converter 110 outputs the image information X-image (X, Y) of the pixels from the top row to the end row. The first memory 207 stores the image information X-image (X, Y).

In step S310, with the X-ray generating apparatus 201 not emitting the X-ray, the drive unit 204 performs, in a period 503, the same controls as the control of the drive lines R1 to R14 of the period 501 of FIGS. 4 and 5 based on the periods TX, TE and the dummy reading stop row RX. More specifically, the impersonation drive is performed.

In step S311, the control unit 202 controls the sensor unit 112 to perform real reading operation 504 of reading out the electric charges of the conversion elements S11 to S33, etc., as in the real reading operation 502. In the real reading operation 504, the drive lines R1 to R14 are sequentially caused to have pulses of the conductive voltages. The switching elements S11 to S33, etc., sequentially enter the conductive state, row by row. Electrical signals are sequentially output to the signal lines Sig1 to Sig3, etc., row by row, from the pixels of the top row to the pixels of the end row. The A/D converter 110 outputs the dark image information Dark_A (X, Y) of the pixels from the top row to the end row. The second memory 208 stores the dark image information Dark_A (X, Y).

FIG. 6 is a diagram illustrating electric charge accumulation periods of the rows of the drive lines R1 to R14 of FIG. 4. The electric charge accumulation period is a period from the last pulse falling time of each of the drive lines R1 to R14 of the period 501 to the pulse rising time of each of the drive lines R1 to R14 of the real reading operation 502. Since the dummy reading of the odd rows is performed after the dummy reading of the even rows, the electric charge accumulation periods of the odd rows and the electric charge accumulation periods of the even rows are different. The electric charge accumulation periods of the even rows R2 and R4 equal to or smaller than the dummy reading stop row RX (=4) are shorter than those of the even rows R6, R8 . . . and R14 greater than the dummy reading stop row RX (=4). Since the dummy reading is sequentially performed row by row, the electric charge accumulation period increases with an increase in the row number.

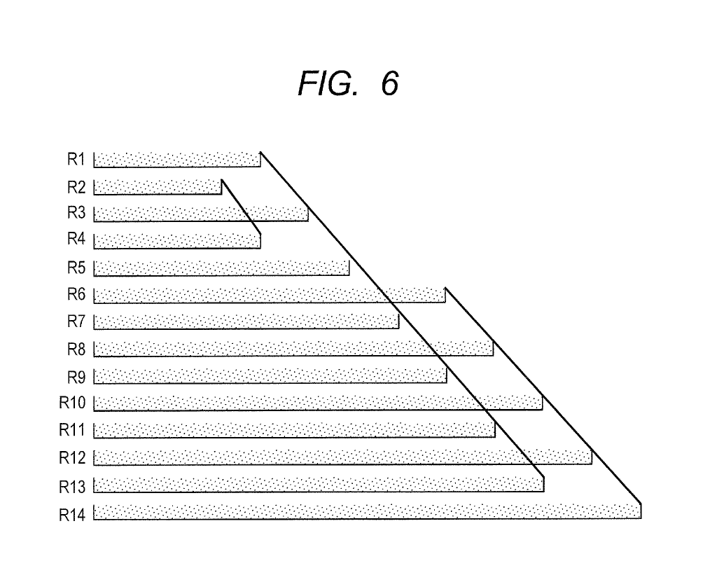

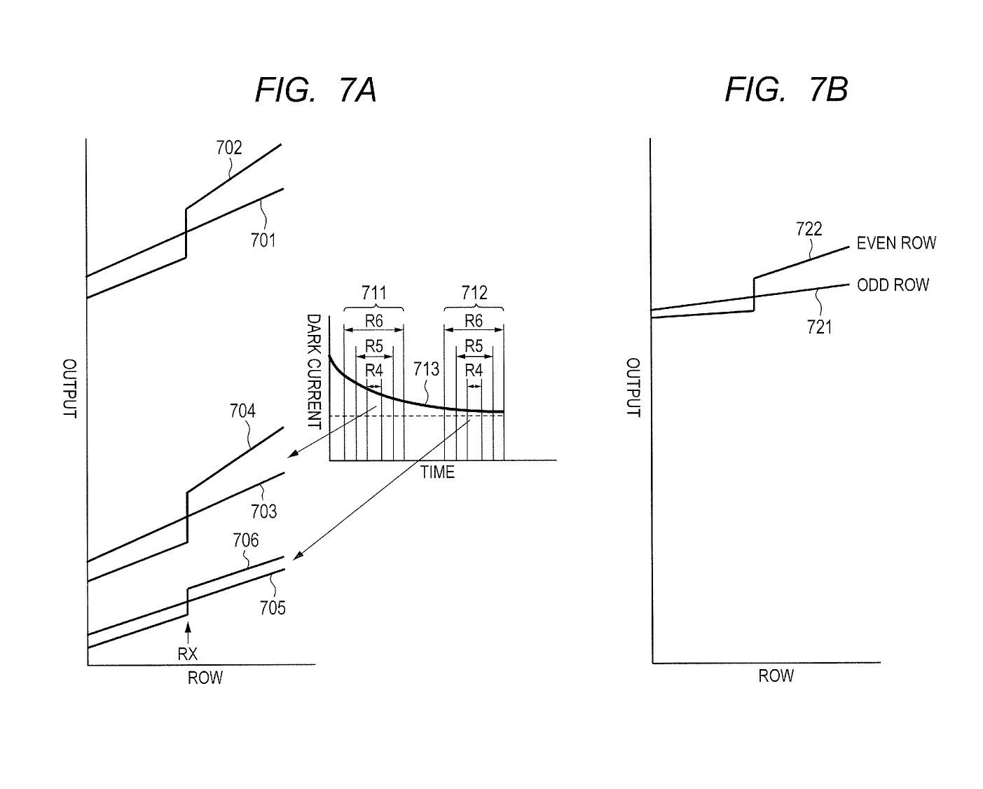

FIG. 7A is a diagram illustrating output values of the rows of the image information X-image (X, Y) and the dark image information Dark_A (X, Y). An output 701 denotes output values of the odd rows of the image information X-image (X, Y). An output 702 denotes output values of the even rows of the image information X-image (X, Y). An output 703 denotes dark current components of the odd rows of the image information X-image (X, Y). An output 704 denotes dark current components of the even rows of the image information X-image (X, Y). An output 705 denotes output values of the odd rows of the dark image information Dark_A (X, Y). An output 706 denotes output values of the even rows of the dark image information Dark_A (X, Y).

A dark current is generated in the sensor unit 112 even in a period that the X-ray is not emitted. Therefore, the noise of the dark current components increases with an increase in the electric charge accumulation period in FIG. 6, and the dark current noise in the image information X-image (X, Y) and the dark image information Dark_A (X, Y) increases. Therefore, dark current noise is generated in the outputs 701 to 706 of FIG. 7A according to the length of the electric charge accumulation period of FIG. 6.

A period 711 corresponds to the period 511 of FIG. 5, and the electric charge accumulation period decreases in order of the sixth row of the drive line R6, the fifth row of the drive line R5 and the fourth row of the drive line R4, as in FIG. 6. The following period 712 corresponds to the period 512 of FIG. 5, and the electric charge accumulation period decreases in order of the sixth row of the drive line R6, the fifth row of the drive line R5 and the fourth row of the drive line R4, as in FIG. 6. A dark current 713 decreases with a lapse of time from the start of the application of the bias voltage Vs. Therefore, the dark current of the period 711 is greater than the dark current of the period 712. As a result, the dark current components 703 and 704 of the image information X-image (X, Y) of the period 711 become greater than the dark image information Dark_A (X, Y) of the period 712.

In step S312 of FIG. 3, the first offset correcting unit 210 subtracts the dark image information Dark_A (X, Y) stored in the second memory 208 from the image information X-image (X, Y) stored in the first memory 207. In this way, the first offset correction is performed.

FIG. 7B is a diagram of output values of the rows illustrating results of the first offset correction. An output 721 denotes a result of the odd rows, wherein the dark image information Dark_A (X, Y) is subtracted from the image information X-image (X, Y). An output 722 denotes a result of the even rows, wherein the dark image information Dark_A (X, Y) is subtracted from the image information X-image (X, Y). If the dark current 713 of FIG. 7A is constant regardless of the time from the start of the application of the bias voltage Vs, the output values of the rows are the same in the outputs 721 and 722 of FIG. 7B. However, the dark current 713 decreases with a lapse of time from the start of the application of the bias voltage Vs, and the result of the first offset correction of FIG. 7B still includes dark current noise. The following second offset correction is performed to remove the dark current noise.

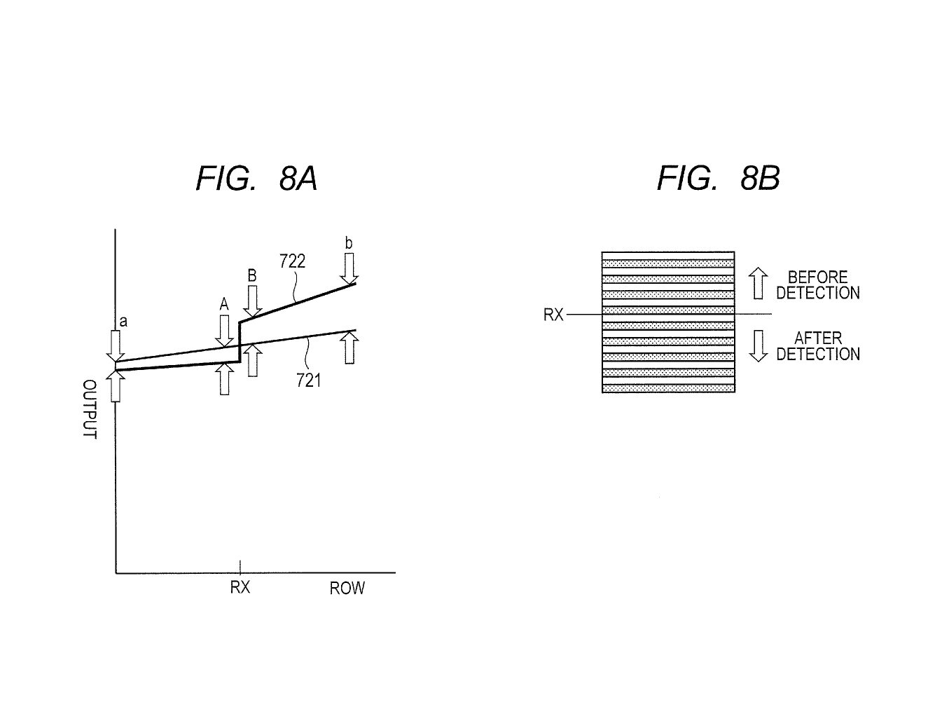

In step S313 of FIG. 3, the second offset correcting unit 211 performs the second offset correction. In the image information corrected by the first offset correcting unit 210, the second offset correcting unit 211 corrects the level difference between the odd rows and the even rows due to the reset operation according to the interlace scanning and the level difference generated before and after the row RX at which the reset operation according to the interlace scanning is stopped. FIGS. 8A to 10B illustrate different examples for realizing step S313 of FIG. 3. The examples will be described in detail below.

FIGS. 8A and 8B are diagrams for describing a first method of the second offset correction. The outputs 721 and 722 of FIG. 8A correspond to the outputs 721 and 722 of FIG. 7B. The output 722 of the even rows is smaller than the output 721 of the odd rows in the even rows equal to or smaller than the dummy reading stop row RX and is greater than the output 721 of the odd rows in the even rows greater than the dummy reading stop row RX. As a result, noise of a stripe artifact as in FIG. 8B is generated in the output image information of the first offset correcting unit 210. The output difference between the odd rows and the even rows may not be constant from the top row to the end row. The output 722 of the even rows has a discontinuity in the dummy reading stop row RX, and the output 721 of the odd rows does not have a discontinuity.

The second offset correcting unit 211 first measures an output difference a between the output 721 of the top odd row and the output 722 of the top odd row as well as an output difference b between the output 721 of the end odd row and the output 722 of the end even row. The second offset correcting unit 211 then measures an output difference A between the output 721 of the odd row just before the dummy reading stop row RX and the output 721 of the even row just before the dummy reading stop row RX as well as an output difference B between the output 721 of the odd row just after the dummy reading stop row RX and the output 722 of the even row just after the dummy reading stop row RX. It should be noted that the output 721 of the odd rows does not have a discontinuity near the stop row RX. Therefore, the second offset correcting unit 211 uses the measured output differences a, A, b and B to perform arithmetic operation processing to bring the output 722 of the odd rows in line with the output 721 of the even rows. A linear correction may be performed, because the amount of level difference continuously changes between the output difference a and the output difference A and between the output difference b and the output difference B. A row average value or an average value of a plurality of pixels can be used to accurately obtain the values of the output differences a, A, b and B. The level difference before and after the dummy reading stop row as well as the even and odd level difference can be reduced with a simple configuration by performing the second offset correction in the method illustrated in FIGS. 8A and 8B.

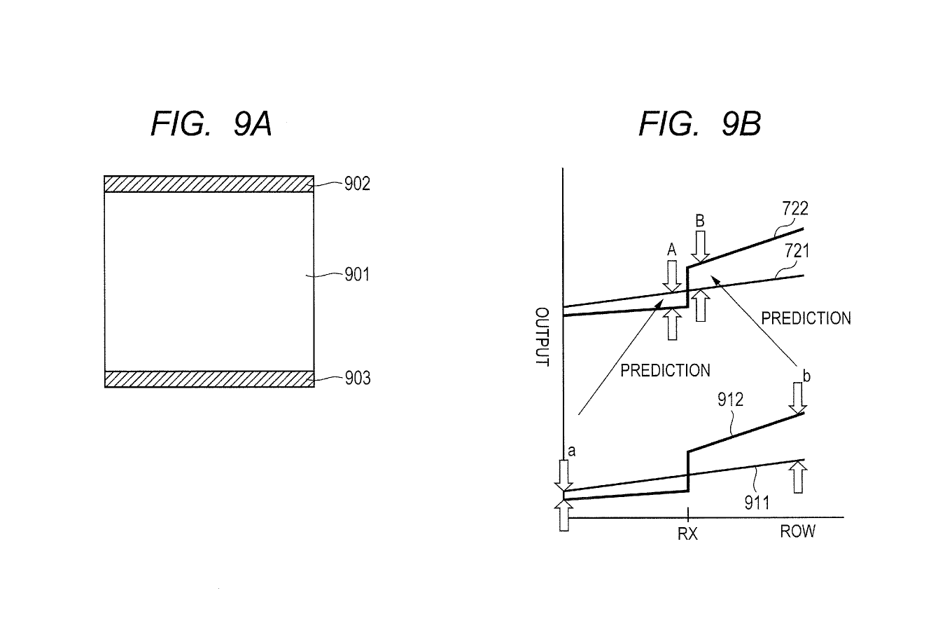

A correction process performed by the second offset correcting unit 211 that is different from FIGS. 8A and 8B will be described with reference to FIGS. 9A and 9B.

FIG. 9A is a diagram illustrating a configuration example of the sensor unit 112 of FIG. 2. The difference from FIGS. 8A and 8B is that the sensor unit 112 includes a normal pixel area 901, a dummy pixel area 902 in the upper end and a dummy pixel area 903 in the lower end. The dummy pixel areas 902 and 903 do not convert the X-ray to electric charges even when the areas are irradiated with the X-ray. The dummy pixel areas 902 and 903 are, for example, light-blocking pixel areas, and the light based on the X-ray is blocked. Therefore, electric charges based on the X-ray are not generated. On the other hand, the normal pixel area 901 does not block light, and an electric charge based on the X-ray is generated. The dummy pixel area 902 in the upper end includes the pixels of the top odd row and the top even row. The dummy pixel area 903 in the lower end includes the pixels of the end odd row and the end even row.

FIG. 9B is a diagram for describing a correction method of the second offset correcting unit 211. As in FIGS. 7B and 8A, the output 721 denotes a result of the odd rows, wherein the dark image information Dark_A (X, Y) is subtracted from the image information X-image (X, Y) when there is no dummy pixel area. The output 722 illustrates a result of the even rows, wherein the dark image information Dark_A (X, Y) is subtracted from the image information X-image (X, Y) when there is no dummy pixel area. Meanwhile, an output 911 denotes an output of the odd rows when all of the pixel areas are assumed to be light-blocking dummy pixel areas, and an output 912 denotes an output of the even rows when all of the pixel areas are assumed to be light-blocking dummy pixel areas. It should be noted that the level difference components due to the dark current are unrelated to the presence/absence of light blocking. More specifically, the difference between the output 722 and the output 721 equals to the difference between the output 912 and the output 911. Therefore, part of the pixel area can be light-blocking as in FIG. 9A to correctly obtain the amount of level difference in the upper end and the amount of level difference in the lower end. The second offset correcting unit 211 first calculates a difference a between the output 911 of the odd rows and the output 912 of the even rows of the dummy pixel area 902 in the upper end, from the output 911 and the output 912 that are the results of the first offset correction. The second offset correcting unit 211 then calculates a difference b between the output 911 of the odd rows and the output 912 of the even rows of the dummy pixel area 903 in the lower end. The second offset correcting unit 211 calculates a difference A between the output 722 of the even rows and the output 721 of the odd rows for each even row based on the dummy reading stop row RX. The second offset correcting unit 211 then calculates a difference B between the output 722 of the even rows and the output 721 of the odd rows based on the dummy reading stop row RX. The second offset correcting unit 211 uses the differences a, A, b and B to perform the correction as described above. In this way, the second offset correction is performed. The second offset correction can prevent noise of a stripe artifact. In the upper end and the lower end of an image, an X-ray not transmitted through the object may be directly irradiated on the pixel area, leading to a so called missing pixel state. In this case, the output may be saturated, and the even and odd level difference components may not be correctly obtained. The dummy pixel areas can be arranged as in FIGS. 9A and 9B to accurately correct the level difference components without being adversely affected by missing pixels.

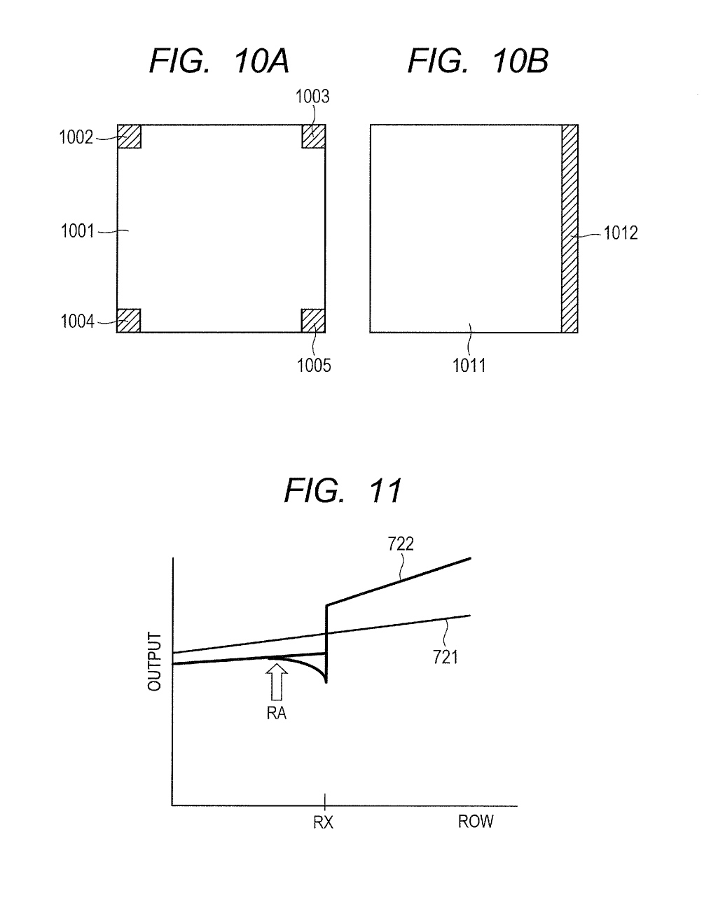

FIG. 10A corresponds to FIG. 9A and is a diagram illustrating an example of other dummy pixel areas 1002 to 1005. The sensor unit 112 includes a normal pixel area 1001, the dummy pixel area 1002 on the upper left, the dummy pixel area 1003 on the upper right, the dummy pixel area 1004 on the lower left and the dummy pixel area 1005 on the lower right. The dummy pixel areas 1002 to 1005 do not convert the X-ray to electric charges even when the areas are irradiated with the X-ray, as in the dummy pixel areas 902 and 903 of FIG. 9A. An electric charge based on the X-ray is generated in the normal pixel area 1001, as in the normal pixel area 901 of FIG. 9A. The dummy pixel areas 1002 and 1003 in the upper end include pixels of the top odd row and the top even row. The dummy pixel areas 1004 and 1005 in the lower end include pixels of the end odd row and the end even row. In this case, the process as described above can also be executed.

FIG. 10B corresponds to FIG. 9A and is a diagram illustrating an example of another dummy pixel area 1012. The sensor unit 112 includes a normal pixel area 1011 and the dummy pixel area 1012 in the right end. The dummy pixel area 1012 does not convert the X-ray to an electric charge even when the area is irradiated with the X-ray, as in the dummy pixel areas 902 and 903 of FIG. 9A. An electric charge based on the X-ray is generated in the normal pixel area 1011, as in the normal pixel area 901 of FIG. 9A. The dummy pixel area 1012 includes the pixels of all rows. In this case, the output difference between the odd rows and the even rows can be detected for all rows to perform the offset correction. In this case, the information of the dummy reading stop row RX may not be necessary.

In step S314 of FIG. 3, the gain correcting unit 212 applies a gain correction to the image information corrected by the second offset correcting unit 211. In FIG. 8A, the output 721 of the odd rows and the output 722 of the even rows indicate lines with gain errors of positive inclinations after the second offset correction of step S313. Therefore, the gain correcting unit 212 corrects the variations of the gains to reduce the gain errors.

The second offset correction of step S313 of FIG. 3 may not be performed if an average value of the differences between the output 721 of the odd rows and the output 722 of the even rows is equal to or smaller than ½ in FIG. 9B. If the period from the bias voltage application start time to the X-ray irradiation start detection time is longer than a predetermined period in FIG. 4, the time change of the dark current is small, and the corrections of steps S313 and S314 of FIG. 3 may not be performed.

In step S315, the artifact correcting unit 213 corrects the artifact. Specifically, the artifact correcting unit 213 corrects the artifact due to the time delay from the detection of the irradiation start of the X-ray by the X-ray detecting unit 203 to the stop of the reset operation according to the interlace scanning, in the image information corrected by the gain correcting unit 212. A case that the X-ray generating apparatus 201 starts the X-ray irradiation after dummy reading of a row RA as illustrated in FIG. 11 will be described. In this case, when the X-ray detecting unit 203 detects the irradiation start of the X-ray, the sensor unit 112 stops the dummy reading operation after dummy reading of the even rows from the row RA to the row RX due to the detection delay of the X-ray detecting unit 203, and the operation shifts to the electric charge accumulating operation. Ideally, when the X-ray generating apparatus 201 starts the X-ray irradiation after the dummy reading of the row RA, the dummy reading operation can be stopped after the dummy reading of the row RA, and the electric charge accumulating operation can be performed. However, the detection and the control of the X-ray detecting unit 203 require a predetermined time, and the dummy reading of the even rows from a row RA+2 to the row RX is performed after the irradiation with the X-ray. This resets the electric charges of the conversion element S41, etc., accumulated by the X-ray irradiation. As a result, the amounts of electric charges of the conversion element S41, etc., accumulated by the X-ray irradiation are smaller in the even rows from the row RA+2 to the row RX than in the other rows. Consequently, the output values of the even rows from the row RA+2 to the row RX are smaller than in the other rows, and a cuneiform artifact is generated. Therefore, the artifact correcting unit 213 corrects the output values of the pixels of the even rows from the row RA+2 to the row RX to reduce the noise of the cuneiform artifact in step S315.

Since the cuneiform artifact exists in the even rows from the row RA+2 to the row RX, the corrections of steps S313 and S314 of FIG. 3 may be performed. In that case, the artifact of the even rows from the row RA+2 to the row RX can be reduced by the correction of step S315.

### Second Embodiment

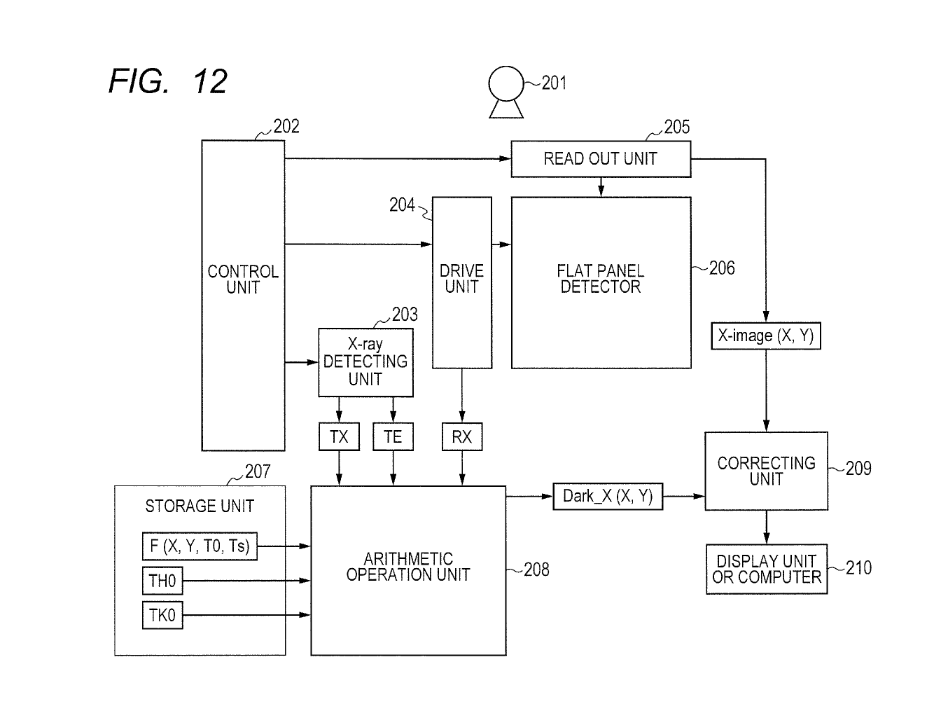

FIG. 12 is a block diagram illustrating a configuration example of a radiation imaging system according to a second embodiment of the present invention.

The radiation imaging system includes an X-ray generating apparatus 201, a control unit 202, an X-ray detecting unit 203, a drive unit 204, a read out unit 205, a flat panel detector 206, a storage unit 207, an arithmetic operation unit 208, a correcting unit 209 and a display unit (or computer) 210. The control unit 202 controls the X-ray detecting unit 203, the drive unit 204 and the read out unit 205. The drive unit 204 drives the flat panel detector 206.

The X-ray generating apparatus (radiation generating apparatus) 201 irradiates the flat panel detector 206 and the X-ray detecting unit 203 with an X-ray (radiation), through an object. The flat panel detector 206 includes a plurality of pixels arranged in a matrix of X columns×Y rows, and each pixel coverts the X-ray transmitted through the object to an electric charge, accumulates the electric charge, and outputs the accumulated electric charge as a pixel output value. The X-ray detecting unit 203 detects an irradiation start and an irradiation end of the X-ray and outputs an irradiation signal of the X-ray to the control unit 202. The flat panel detector 206 starts accumulating the electric charge based on the X-ray when the X-ray detecting unit 203 detects the irradiation start of the X-ray, and the flat panel detector 206 outputs the pixel output value based on the accumulated electric charge to the read out unit 205 when the X-ray detecting unit 203 detects the irradiation end of the X-ray. Consequently, the read out unit 205 outputs image information X-image (X, Y) based on the X-ray irradiation of the pixels of X columns×Y rows.

The X-ray detecting unit 203 outputs times TX and TE to the arithmetic operation unit 208. The time TX is a time from a bias voltage application start time to an X-ray irradiation start detection time as illustrated in FIG. 4. The time TE is a time from the bias voltage application start time to an X-ray irradiation end detection time as illustrated in FIG. 4. The drive unit 204 outputs a row RX, at which dummy reading (reset operation) is stopped, to the arithmetic operation unit 208 at the X-ray irradiation start, as illustrated in FIG. 4.

The storage unit 207 stores, in advance, dark current characteristics F (X, Y, T0, Ts) of the pixels, a time TH0 for one row of the real reading (read out operation) of FIG. 4 and a time TK0 for one row of the dummy reading (reset operation) of FIG. 4 and outputs them to the arithmetic operation unit 208. The arithmetic operation unit 208 inputs the dark current characteristics F (X, Y, t0, Ts), the time TH0, the time TK0, the time TX, the time TE and the dummy reading stop row RX to compute and predict dark current components Dark_X (X, Y) superimposed on the signal of each pixel in the X-ray imaging. Specifically, the arithmetic operation unit 208 calculates an electric charge accumulation start time T0 and an electric charge accumulation time Ts of each row of FIG. 4 based on the time TH0, the time TK0, the time TX, the time TE and the dummy reading stop row RX. Thereafter, the arithmetic operation unit 208 calculates the dark current characteristics F (X, Y, T0, Ts) based on the electric charge accumulation start time T0 and the electric charge accumulation time Ts to predict the dark current components Dark_X (X, Y) contained in each pixel in the X-ray imaging. The correcting unit 209 subtracts the dark current components Dark_X (X, Y) from the image information X-image (X, Y) to correct the image information. The display unit (or computer) 210 displays (or processes) the image information corrected by the correcting unit 209.

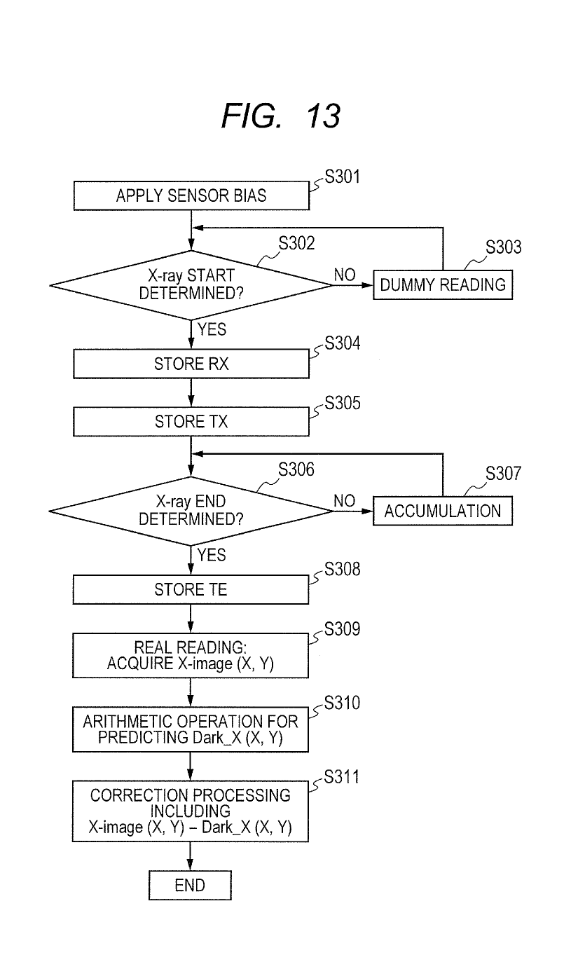

FIG. 13 is a flow chart illustrating a control method of the radiation imaging system of FIG. 1. In step S301, the current-voltage conversion circuit 115 starts applying the bias voltage Vs to the bias line Bs as illustrated in FIG. 4.

In step S302, the control unit 202 determines whether irradiation with an X-ray is started. The X-ray detecting unit 203 outputs an X-ray irradiation signal to the control unit 202 if the current information (electrical signal based on the emitted X-ray) output by the A/D converter 127 is equal to or greater than a threshold. The control unit 202 determines that the irradiation with the X-ray is started if the X-ray irradiation signal is input and determines that the irradiation with the X-ray is not started if the X-ray irradiation signal is not input. The process proceeds to step S304 if the irradiation with the X-ray is started and proceeds to step S303 if the irradiation with the X-ray is not started. In step S303, the control unit 202 controls the sensor unit 112 to set the drive lines R1 to R14 to the conductive voltages and to cause the switching elements T11 to T33, etc., to enter the conductive state as illustrated in FIG. 4. In this way, reset operation (hereinafter, called “dummy reading”) of resetting the electric charges of the conversion elements S11 to S33, etc., generated by electric charge accumulation of a dark current is performed. Thereafter, the process returns to step S302. Before the irradiation with the X-ray, the sensor unit 112 repeats the reset operation of resetting the electric charges of the conversion elements S11 to S33, etc., caused by the dark current.

In the dummy reading of step S303, reset operation according to interlace scanning is performed as illustrated in FIG. 4. First, the drive lines R2, R4, R6 . . . and R14 of the pixels of the even rows are sequentially set to the conductive voltages. The switching elements T21, T41, etc., of the pixels of the even rows sequentially enter the conductive state. The electric charges of the conversion elements S21, S41, etc., of the pixels of the even rows are reset. Next, the drive lines R1, R3, R5 . . . and R13 of the pixels of the odd rows are sequentially set to the conductive voltages. The switching elements T11, T31, etc. of the pixels of the odd rows sequentially enter the conductive state. The electric charges of the conversion elements S11, S31, etc., of the pixels of the odd rows are reset. The operation of the set of the reset of the even rows and the reset of the odd rows is repeated until the detection of the start of the X-ray.

After the detection of the X-ray irradiation start, the drive unit 204 stores the X-ray detected row RX in step S304. The X-ray detected row RX denotes the row RX, at which the dummy reading is stopped due to the X-ray irradiation start, and is a fourth row corresponding to the drive line R4 in the case of FIG. 4.

In step S305, the drive unit 204 inputs the time TX from the X-ray detecting unit 203 through the control unit 202 and stores the time TX. As illustrated in FIG. 4, the time TX is a time from the bias voltage application start time to the X-ray irradiation start detection time.

In step S306, the control unit 202 determines whether the irradiation with the X-ray is finished. If the current information (electrical signal based on the emitted X-ray) output by the A/D converter 127 is smaller than the threshold, the X-ray detecting unit 203 stops outputting the X-ray irradiation signal. The X-ray detecting unit 203 may stop outputting the X-ray irradiation signal after a lapse of a predetermined time (X-ray irradiation period) from the detection time of the X-ray irradiation start. The control unit 202 determines that the irradiation with the X-ray is finished if the input of the X-ray irradiation signal is stopped and determines that the irradiation with the X-ray is not finished if the X-ray irradiation signal is input. The process proceeds to step S308 if the irradiation with the X-ray is finished and proceeds to step S307 if the irradiation with the X-ray is not finished. In step S307, the control unit 202 controls the sensor unit 112 to perform accumulating operation of electric charges. Thereafter, the process returns to step S306. As illustrated in FIG. 4, the accumulating operation of the electric charges is operation of setting all of the drive lines R1 to R14 to the non-conductive voltages and causing the switching elements T11 to T33, etc., of all of the pixels to enter the non-conductive state to accumulate the electric charges based on the irradiation with the X-ray in the conversion elements S11 to S33, etc. The accumulating operation of the electric charges is performed in the conversion elements S11 to S33, etc., until the end of the irradiation with the X-ray.

After the detection of the X-ray irradiation end, the control unit 204 inputs the time TE from the X-ray detecting unit 203 through the control unit 202 in step S308 and stores the time TE. As illustrated in FIG. 4, the time TE is a time from the bias voltage application start time to the X-ray irradiation end time.

In step S309, the control unit 202 controls the sensor unit 112 to perform real reading operation 502 of reading out the electric charges based on the irradiation with the X-ray. In the real reading operation 502, the drive lines R1 to R14 are sequentially caused to have pulses of the conductive voltages. The switching elements S11 to S33, etc., sequentially enter the conductive state, row by row. Electrical signals are sequentially output to the signal lines Sig1 to Sig3, etc., row by row, from the pixels of the top row to the pixels of the end row. The A/D converter 110 outputs the image information X-image (X, Y) of the pixels from the top row to the end row. The correcting unit 209 inputs the image information X-image (X, Y).

In step S310, the arithmetic operation unit 208 calculates the electric charge accumulation start time T0 and the electric charge accumulation time Ts of each row of FIG. 4 based on the time TH0, the time TK0, the time TX, the time TE and the dummy reading stop row RX. Thereafter, the arithmetic operation unit 208 calculates the dark current characteristics F (X, Y, T0, Ts) based on the electric charge accumulation start time T0 and the electric charge accumulation time Ts to predict the dark current components Dark_X (X, Y) contained in each pixel in the X-ray imaging.

In step S311, the correcting unit 209 subtracts the dark current components Dark_X (X, Y) from the image information X-image (X, Y) to correct the image information. The display unit (or computer) 210 displays (or processes) the image information corrected by the correcting unit 209.

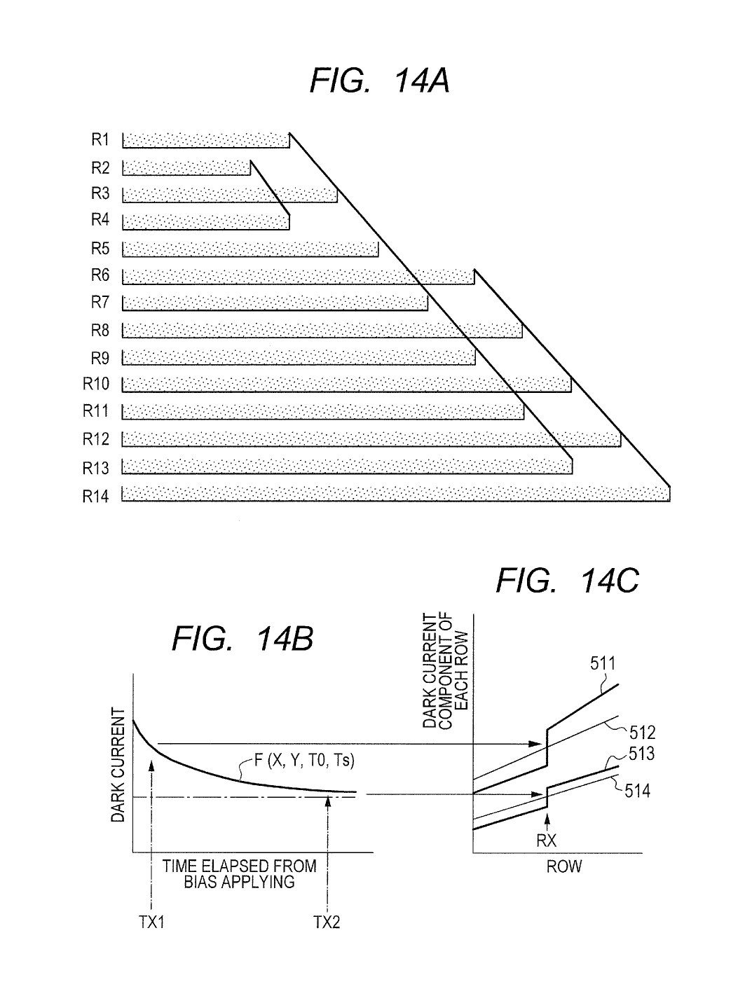

FIG. 14A is a diagram illustrating electric charge accumulation periods of the rows of the drive lines R1 to R14 of FIG. 4. The electric charge accumulation period is a period from the last pulse falling time of each of the drive lines R1 to R14 of the period 501 to the pulse rising time of each of the drive lines R1 to R14 of the real reading operation 502. Since the dummy reading of the odd rows is performed after the dummy reading of the even rows, the electric charge accumulation periods of the odd rows and the electric charge accumulation periods of the even rows are different. The electric charge accumulation periods of the even rows R2 and R4 equal to or smaller than the dummy reading stop row RX (=4) are shorter than those of the even rows R6, R8 . . . and R14 greater than the dummy reading stop row RX (=4). Since the dummy reading is sequentially performed row by row, the electric charge accumulation period increases with an increase in the row number.

FIG. 14B is a diagram illustrating the dark current characteristics F (X, Y, T0, Ts) relative to the time elapsed from the application of bias, and FIG. 14C is a diagram illustrating the dark current components Dark_X (X, Y) of each row. The dark current characteristics F (X, Y, T0, Ts) decrease with an increase in the time elapsed from the application of bias. If an elapsed time TX2 from the application of bias is longer than an elapsed time TX1 from the application of bias, the dark current characteristics F (X, Y, T0, Ts) at the elapsed time TX2 are smaller than the dark current characteristics F (X, Y, T0, Ts) at the elapsed time TX1.

Dark current components 511 denote the dark current components Dark_X (X, Y) of the even rows at the elapsed time TX1. Dark current components 512 denote the dark current components Dark_X (X, Y) of the odd rows at the elapsed time TX1. Dark current components 513 denote dark current components Dark_X (X, Y) of the even rows at the elapsed time TX2. Dark current components 514 denote the dark current components Dark_X (X, Y) of the odd rows at the elapsed time TX2.

In the sensor unit 112, the dark current is generated even in a period without the irradiation with the X-ray. Therefore, the dark current components increase with an increase in the electric charge accumulation period in FIG. 14A. Thus, the sizes of the rows of the dark current components 511 to 514 of FIG. 14C correspond to the length of the electric charge accumulation period of FIG. 14A. The dark current components 511 and 513 of the even rows have level differences in the dummy reading stop row RX. Therefore, the correcting unit 209 can subtract the dark current components Dark_X (X, Y) from the image information X-image (X, Y) to correct the image information to obtain a high-quality image.

### Third Embodiment

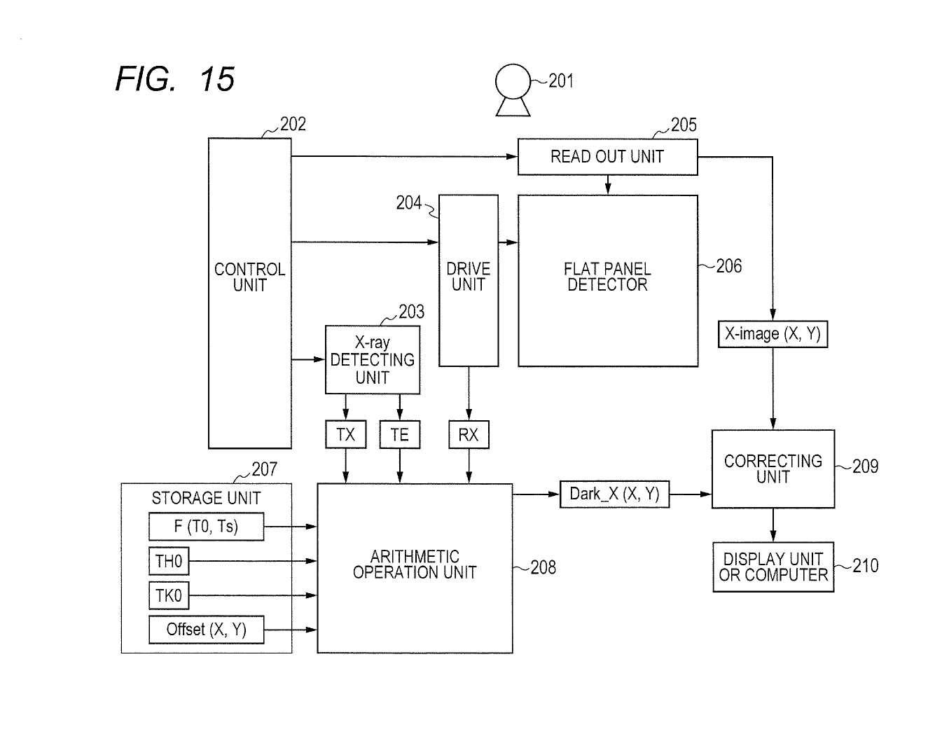

FIG. 15 is a block diagram illustrating a configuration example of a radiation imaging system according to a third embodiment of the present invention. In FIG. 15, the storage content of the storage unit 207 is different from FIG. 12. Hereinafter, the difference from the second embodiment in the present embodiment will be described. In place of the dark current characteristics F (X, Y, T0, Ts) of each pixel in the storage unit 207 of FIG. 12, the storage unit 207 of FIG. 15 stores, in advance, dark current characteristics F (T0, Ts) common to all of the pixels and offset values Offset (X, Y) of each pixel. The dark current characteristics F (T0, Ts) denote dark current characteristics common to all of the pixels, such as an average of all of the pixels. The offset values Offset (X, Y) denote offset values of each pixel not including the dark current components. The arithmetic operation unit 208 can use the dark current characteristics F (T0, Ts) common to all of the pixels and the offset values Offset (X, Y) of each pixel in place of the dark current characteristics F (X, Y, T0, Ts) of each pixel to predict the dark current components Dark_X (X, Y). The correcting unit 209 subtracts the dark current components Dark_X (X, Y) from the image information X-image (X, Y) to correct the image information. According to the present embodiment, a high-quality image can be obtained while reducing the storage capacity of the storage unit 207 and the operation load of the arithmetic operation unit 208.

### Fourth Embodiment

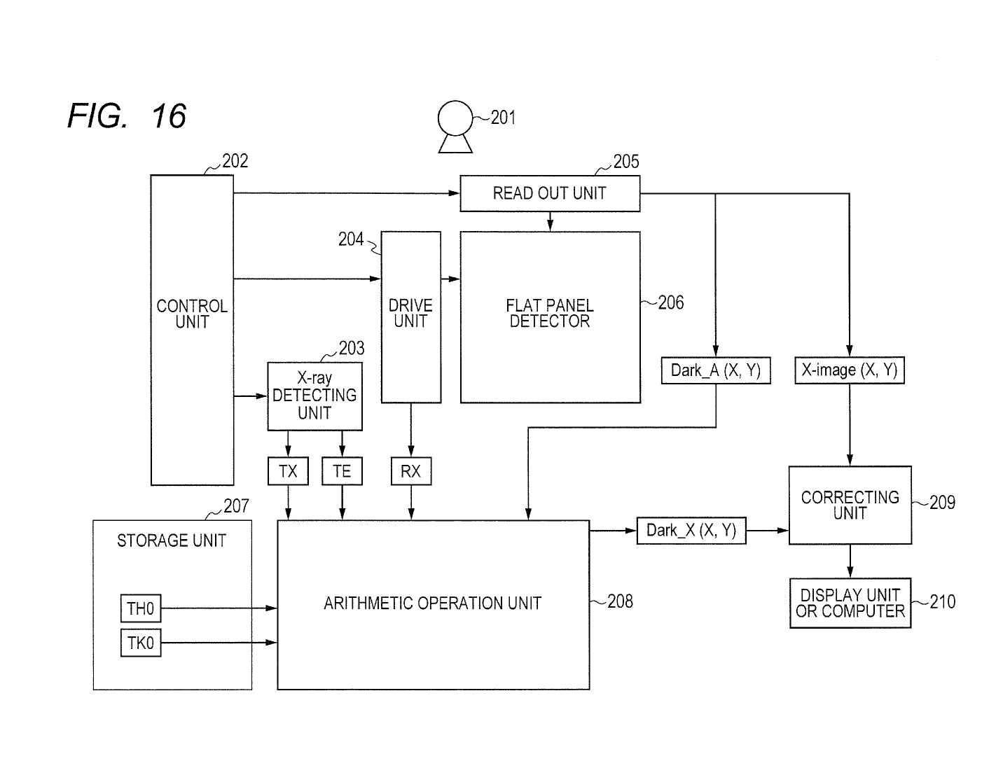

FIG. 16 is a block diagram illustrating a configuration example of a radiation imaging system according to a fourth embodiment of the present invention. Hereinafter, the difference from the second embodiment in the present embodiment will be described. The read out unit 205 outputs, from the flat panel detector 206, fixed pattern noise information Dark_A (X, Y) in addition to the image information X-image (X, Y). The storage unit 207 stores, in advance, the time TH0 for one row of real reading and the time TK0 for one row of dummy reading and outputs them to the arithmetic operation unit 208. The arithmetic operation unit 208 inputs the time TH0, the time TK0 and the fixed pattern noise information Dark_A (X, Y) to compute and predict the dark current components Dark_X (X, Y) superimposed on the signal of each pixel in the X-ray imaging. The correcting unit 209 subtracts the dark current components Dark_X (X, Y) from the image information X-image (X, Y) to correct the image information. The display unit (or computer) 210 displays (or processes) the image information corrected by the correcting unit 209.

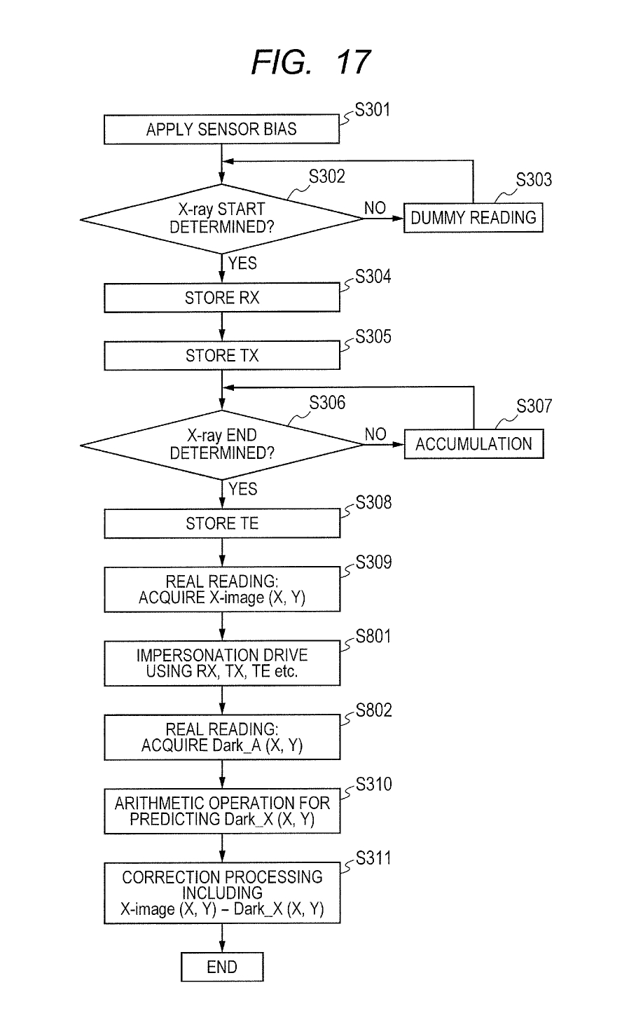

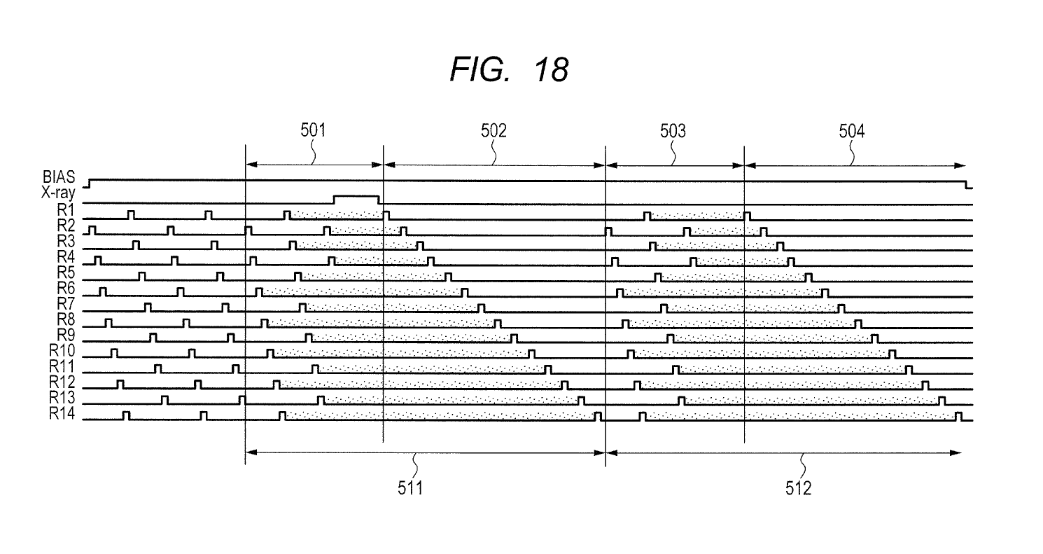

FIG. 17 is a flow chart illustrating a control method of the radiation imaging system of FIG. 16, and FIG. 18 is a timing chart of the control method. The early part of FIG. 18 is the same as FIG. 4. In FIG. 17, steps S801 and S802 are added between steps S309 and S310 of FIG. 13. Hereinafter, the difference from FIG. 13 in FIG. 17 will be described.

In step S801 after step S309, the drive unit 204 performs, in the period 503, the same control as the control of the drive lines R1 to R14 in the period 501 of FIGS. 4 and 18 based on the times TX, TE and the dummy reading stop row RX, with the X-ray generating apparatus 201 not emitting the X-ray. More specifically, the impersonation drive is performed.

In step S802, the control unit 202 controls the sensor unit 112 to perform the real reading operation 504 of reading out the electric charges of the conversion elements S11 to S33, etc., as in the real reading operation 502. In the real reading operation 504, the drive lines R1 to R14 are sequentially caused to have pulses of the conductive voltages. The switching elements S11 to S33, etc., sequentially enter the conductive state, row by row. Electrical signals are sequentially output to the signal lines Sig1 to Sig3, etc., row by row, from the pixels of the top row to the pixels of the end row. The A/D converter 110 outputs the fixed pattern noise information Dark_A (X, Y) of the pixels from the top row to the end row. The arithmetic operation unit 208 inputs the fixed pattern noise information Dark_A (X, Y).

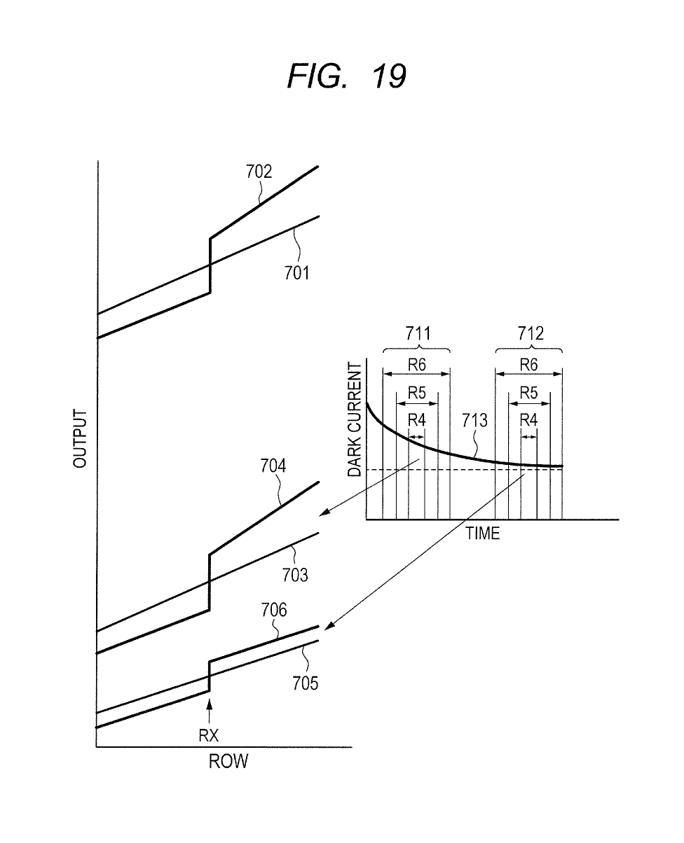

FIG. 19 is a diagram illustrating output values of the rows of the image information X-image (X, Y) and the fixed pattern noise information Dark_A (X, Y), and FIG. 19 corresponds to FIG. 14A. An output 701 denotes output values of the odd rows of the image information X-image (X, Y). An output 702 denotes output values of the even rows of the image information X-image (X, Y). An output 703 denotes dark current components of the odd rows of the image information X-image (X, Y). An output 704 denotes dark current components of the even rows of the image information X-image (X, Y). An output 705 denotes output values of the odd rows of the fixed pattern noise information Dark_A (X, Y). An output 706 denotes output values of the even rows of the fixed pattern noise information Dark_A (X, Y).

A dark current is generated in the sensor unit 112 even in a period that the X-ray is not emitted. Therefore, the noise of the dark current components increases with an increase in the electric charge accumulation period in FIG. 14A, and the dark current noise in the image information X-image (X, Y) and the fixed pattern noise information Dark_A (X, Y) increases. Therefore, dark current noise is generated in the outputs 701 to 706 of FIG. 19 according to the length of the electric charge accumulation period of FIG. 14A. The outputs 702, 704 and 706 of the even rows have level differences in the dummy reading stop row RX.

A period 711 corresponds to the period 511 of FIG. 18, and the electric charge accumulation period decreases in order of the sixth row of the drive line R6, the fifth row of the drive line R5 and the fourth row of the drive line R4, as in FIG. 14A. The following period 712 corresponds to the period 512 of FIG. 18, and the electric charge accumulation period decreases in order of the sixth row of the drive line R6, the fifth row of the drive line R5 and the fourth row of the drive line R4, as in FIG. 14A. A dark current 713 decreases with a lapse of time. Therefore, the dark current of the period 711 is greater than the dark current of the period 712. As a result, the dark current components 703 and 704 of the image information X-image (X, Y) of the period 711 become greater than the outputs 705 and 706 of the fixed pattern noise information Dark_A (X, Y) of the period 712.

In step S310 of FIG. 17, the arithmetic operation unit 208 predicts the dark current components Dark_X (X, Y) contained in each pixel in the X-ray imaging based on the time TH0, the time TK0 and the fixed pattern noise information Dark_A (X, Y).

In step S311, the correcting unit 209 subtracts the dark current components Dark_X (X, Y) from the image information X-image (X, Y) to correct the image information. The display unit (or computer) 210 displays (or processes) the image information corrected by the correcting unit 209. According to the present embodiment, the image information can be more accurately corrected, and excellent image quality can be obtained.

### Fifth Embodiment

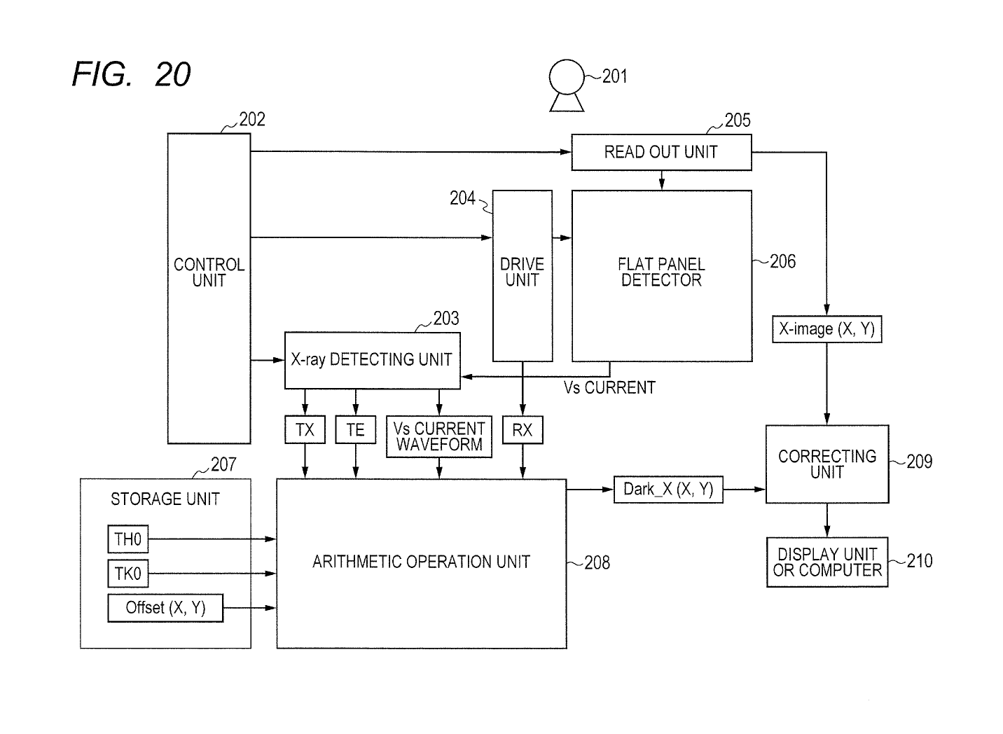

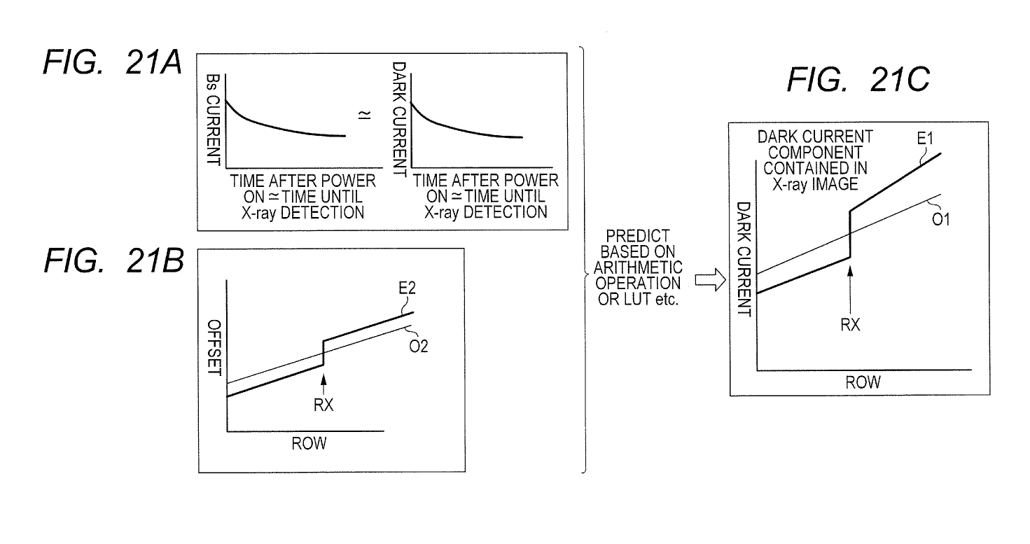

FIG. 20 is a block diagram illustrating a configuration example of a radiation imaging system according to a fifth embodiment of the present invention. FIG. 20 is different from FIG. 15 in that current waveform information of the bias line Bs is input instead of the input of the representative dark current characteristics F (T0, Ts) by the arithmetic operation unit 208. The present inventors have found out that the current waveform information of the bias line Bs resembles the dark current characteristics F (T0, Ts) as illustrated in FIG. 21A. Therefore, the current waveform information of the bias line Bs is used in place of the dark current characteristics F (T0, Ts). Hereinafter, the difference from FIG. 15 in FIG. 20 will be described.

As described, the X-ray detecting unit 203 inputs the current information (electrical signal based on the emitted X-ray) flowing in the bias line Bs of all of the pixels of FIG. 2, from the A/D converter 127 (FIG. 2) in the flat panel detector 206. The X-ray detecting unit 203 outputs the current waveform information of the bias line Bs to the arithmetic operation unit 208 based on the current information of the bias line Bs.

The storage unit 207 stores, in advance, the time TH0 for one row of real reading, the time TK0 for one row of dummy reading and the offset values Offset (X, Y) of each pixel and outputs them to the arithmetic operation unit 208. As illustrated in FIG. 21C, an offset value O2 of the odd rows and an offset value E2 of the even rows are different in the offset values Offset (X, Y) of each pixel as in FIG. 19. The offset value E2 of the even rows has a level difference in the dummy reading stop row RX. The arithmetic operation unit 208 inputs the time TH0, the time TK0, the offset values Offset (X, Y), the time TX, the time TE, the current waveform information of the bias line Bs and the dummy reading stop row RX. As illustrated in FIG. 21C, the arithmetic operation unit 208 performs arithmetic operation of the dark current components Dark_X (X, Y) superimposed on the signal of each pixel in the X-ray imaging or uses a look-up table to predict the dark current components Dark_X (X, Y), based on the inputs. Dark current components O1 of the odd rows and dark current components E1 of the even rows are difference in the dark current components Dark_X (X, Y). The dark current components E1 of the even rows have a level difference in the dummy reading stop row RX. Since the current waveform information of the bias line Bs resembles the dark current characteristics F (T0, Ts), the arithmetic operation unit 208 uses the current waveform information of the bias line Bs in place of the dark current characteristics F (T0, Ts) of FIG. 15 to predict the dark current components Dark_X (X, Y). The correcting unit 209 subtracts the dark current components Dark_X (X, Y) from the image information X-image (X, Y) to correct the image information. The display unit (or computer) 210 displays (or processes) the image information corrected by the correcting unit 209.

In the present embodiment, the resemblance of the current waveform information of the bias line Bs to the dark current characteristics F (T0, Ts) can be used to highly accurately predict the dark current components Dark_X (X, Y) even if the dark current components Dark_X (X, Y) vary due to temperature, etc. The present embodiment has advantages that the storage capacity of the storage unit 207 and the operation load of the arithmetic operation unit 208 can be reduced and that changes in the dark current characteristics caused by the temperature or variations (degradations) in characteristics can be handled.

### Sixth Embodiment

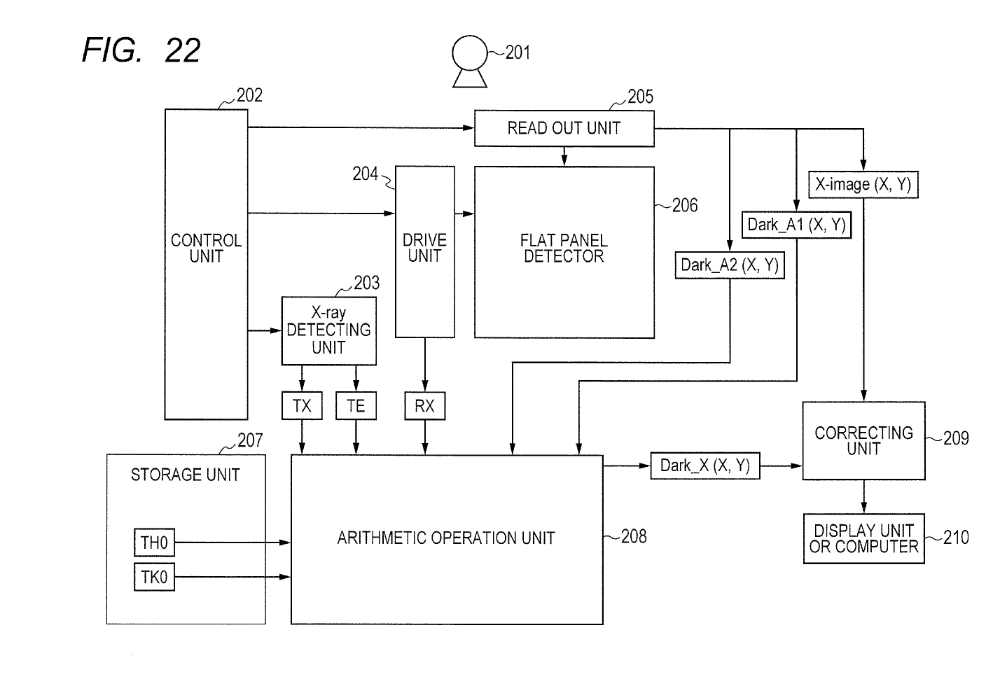

FIG. 22 is a block diagram illustrating a configuration example of a radiation imaging system according to a sixth embodiment of the present invention. FIG. 22 is different from FIG. 16 in that the arithmetic operation unit 208 inputs first fixed pattern noise information Dark_A1 (X, Y) and second fixed pattern noise information Dark_A2 (X, Y) from the read out unit 205. Hereinafter, the difference from the fourth embodiment (FIG. 16) in the present embodiment (FIG. 22) will be described.

In the present embodiment, processes of steps S801 and S802 are executed in FIG. 17 to acquire the first fixed pattern noise information Dark_A1 (X, Y). Thereafter, the processes of steps S801 and S802 are executed again to acquire the second fixed pattern noise information Dark_A2 (X, Y).

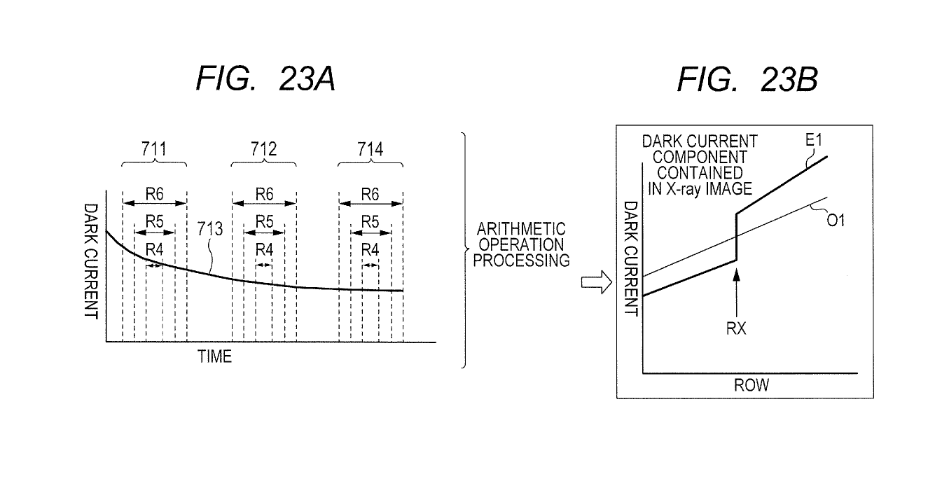

FIG. 23A corresponds to FIG. 19 and is a diagram illustrating the dark current 713 relative to the time. The period 711 corresponds to the period 511 of FIG. 18 and denotes a dark current contained in the image information X-image (X, Y) of step S309 of FIG. 17. The following period 712 corresponds to the period 512 of FIG. 18 and corresponds to the first fixed pattern noise information Dark_A1 (X, Y) of the first step S802 of FIG. 17. The following period 714 corresponds to the second fixed pattern noise information Dark_A2 (X, Y) of the second step S802 of FIG. 17, and the electric charge accumulation period decreases in order of the sixth row of the drive line R6, the fifth row of the drive line R5 and the fourth row of the drive line R4. The dark current 713 decreases with a lapse of time. Therefore, the dark current of the period 714 is smaller than the dark current of the period 712.

Thereafter, the arithmetic operation unit 208 inputs the time TH0, the time TK0, the time TX, the time TE and the dummy reading stop row RX in step S310 of FIG. 17. The arithmetic operation unit 208 further inputs the first fixed pattern noise information Dark_A1 (X, Y) of the period 712 and the second fixed pattern noise information Dark_A2 (X, Y) of the period 714. As illustrated in FIGS. 23A and 23B, the arithmetic operation unit 208 computes and predicts the dark current components Dark_X (X, Y) contained in each pixel in the X-ray imaging based on the inputs. The dark current components O1 of the odd rows and the dark current components E1 of the even rows are different in the dark current components Dark_X (X, Y). The dark current components E1 of the even rows have a level difference in the dummy reading stop row RX. The arithmetic operation unit 208 computes an amount of change in the dark current based on the difference between the first fixed pattern noise information Dark_A1 (X, Y) of the period 712 and the second fixed pattern noise information Dark_A2 (X, Y) of the period 714 to compute the dark current components Dark_X (X, Y) based on the amount of change.

In step S311 of FIG. 17, the correcting unit 209 subtracts the dark current components Dark_X (X, Y) from the image information X-image (X, Y) to correct the image information. The display unit (or computer) 210 displays (or processes) the image information corrected by the correcting unit 209. The present embodiment has an advantage that the storage capacity of the storage unit 207 and the operation load of the arithmetic operation unit 208 can be reduced.

The above-described embodiments are intended to illustrate examples for implementing the present invention and should not be construed as limiting the technical scope of the present invention. The present invention can be implemented in various forms without departing from the technical concept and main features of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2013-118994, filed Jun. 5, 2013, and No. 2013-143815, filed Jul. 9, 2013, which are hereby incorporated by reference herein in their entirety.

What is claimed is:

1. A radiation imaging system comprising:
a sensor unit including a plurality of pixels arranged in a matrix, and configured to convert a radiation into an electric charge and to output a pixel output value;
a detecting unit configured to detect an irradiation start of the radiation;
a control unit to perform (A) a first reset operation (A1) for resetting the plurality of pixels according to an interlace scanning until the detection of the irradiation start of the radiation by the detecting unit, a first accumulating operation (A2) for accumulating the electric charge in response to the detection of the irradiation start of the radiation by the detecting unit by stopping the first reset operation, and, thereafter, a first read out operation (A3) for reading out a pixel output value of the plurality of pixels in response to an irradiation end of the radiation so as to output an image information based on the irradiation of the radiation, and to further perform (B) a second reset operation (B1) for resetting the plurality of pixels according to the interlace scanning after the first read out operation, a second accumulating operation (B2) for accumulating the electric charge by stopping the second reset operation and a second read out operation (B3) for reading out the pixel output value so as to output a dark image information in accordance with the second accumulating operation, wherein the operations (A1), (A2), (A3), (B1), (B2) and (B3) are performed in this order;
a first correcting unit configured to perform a correction by subtracting the dark image information from the image information based on the irradiation of the radiation;
a second correcting unit configured to perform a correction of the image information after the correction by the first correcting unit, so as to correct (1) a level shift formed in the image information between an odd row and an even row of the pixels due to the interlace reset operation and to correct (2) a level shift formed in the image information between a row of the pixels scanned before stopping the interlace reset operation and a row of the pixels scanned after stopping the interlace reset operation.

2. The radiation imaging system according to claim 1, wherein
the sensor unit a plurality of dummy pixels configured not to convert the radiation into the electric charge,
the second correcting unit performs the correction using a pixel value of the plurality of dummy pixels.

3. The radiation imaging system according to claim 2, wherein
the dummy pixels are arranged at least in top odd and even rows and end odd and even rows.

4. The radiation imaging system according to claim 1, further comprising
a gain correcting unit configured to subject, to a gain correction, the image information after the correction by the second correcting unit.

5. The radiation imaging system according to claim 1, further comprising
an artifact correcting unit configured to correct an artifact of the image information after the correction by the second correcting unit, the artifact is due to a time delay from the detection of the irradiation start of the radiation by the detecting unit until the stopping the reset operation according to the interlace scanning.

6. The radiation imaging system according to claim 1, further comprising
a radiation generating apparatus configured to irradiate the radiation.

7. A radiation imaging system comprising:
a sensor unit including a plurality of pixels arranged in a matrix, and configured to convert a radiation into an electric charge and to output a pixel output value;
a detecting unit configured to detect an irradiation start of the radiation and an irradiation end of the radiation;
a control unit to perform a reset operation (1) for resetting the plurality of pixels according to an interlace scanning until the detection of the irradiation start of the radiation by the detecting unit, a accumulating operation (2) for accumulating the electric charge in response to the detection of the irradiation start of the radiation by the detecting unit by stopping the first reset operation, and, thereafter, a read out operation (3) for reading out a pixel output value of the plurality of pixels in response to an irradiation end of the radiation so as to output an image information based on the irradiation of the radiation, wherein the operations (1), (2) and (3) are performed in this order;
an arithmetic operation unit configured to calculate a dark current component based on a period of the reset operation (1) for one row, a period of the read out operation (3) for one row, a period of detecting the irradiation start of the radiation by the detecting unit, a period of detecting the irradiation end of the radiation by the detecting unit, and a row at which the reset operation is stopped; and
a correcting unit configured to correct the image information based on the dark current component.

8. The radiation imaging system according to claim 7, further comprising
the arithmetic operation unit calculates the dark current component based on a dark current characteristic of each of the pixels.

9. The radiation imaging system according to claim 7, further comprising
the arithmetic operation unit calculates the dark current component based on a dark current characteristic common to all of the pixels, and an offset value of each of the pixels.

10. The radiation imaging system according to claim 7, wherein,
the control unit further performs, after outputting the image information, (B) the reset operation (1) for resetting the plurality of pixels according to the interlace scanning, the accumulating operation (2) for accumulating the electric charge, and the read out operation (3) for reading out the pixel output value, so as to output a first fixed pattern noise information, and
the arithmetic operation unit calculates the dark current component based on the first fixed pattern noise information.

11. The radiation imaging system according to claim 7, wherein,
the arithmetic operation unit calculates the dark current component based on a current flowing in a bias line of the plurality of pixels and an offset value of each of the pixels.

12. The radiation imaging system according to claim 10, wherein,
the control unit further performs, after outputting the first fixed pattern noise information, (B) the reset operation (1) for resetting the plurality of pixels according to the interlace scanning, the accumulating operation (2) for accumulating the electric charge, and the read out operation (3) for reading out the pixel output value, so as to output a second fixed pattern noise information, and
the arithmetic operation unit calculate the dark current component based on the first fixed pattern noise information and the second fixed pattern noise information.

13. The radiation imaging system according to claim 7, further comprising
a radiation generating apparatus configured to irradiate the radiation.
